(19) United States
(12) Patent Application Publication
(10) Pub. No.: US 2011/0126325 A1
(43) Pub. Date: May 26, 2011

(54) RECOMBINANT CELLS AND METHODS FOR HYDROXYLATING FATTY ACIDS

(75) Inventors: Xue-Rong Zhou, Evatt (AU); Surinder Pal Singh, Downer (AU); Allan Green, Red Hill (AU); Anthony Richard Ashton, Cook (AU); Andrew John William Rodgers, Richmond (AU); Geoffrey John Dumsday, Vermont South (AU); Florian Hans Maximilian Graichen, Glen Iris (AU)

(73) Assignees: Commonwealth Scientific Industrial Research Organisation; Grains Research and Development Corporation

(21) Appl. No.: 12/989,377

(22) PCT Filed: Apr. 24, 2009
(86) PCT No.: PCT/AU09/00518

§ 371 Date: Feb. 8, 2011

Related U.S. Application Data
(60) Provisional application No. 61/125,437, filed on Apr. 25, 2008.

(51) Int. Cl. A01H 5/10; C12Q 1/68; C12Q 1/02; C12P 1/00; C12P 7/64; C12N 9/02; C12N 5/04; C12N 1/00; C12N 15/63; C07H 21/00; C12N 15/53; C07C 57/00

(52) U.S. Cl. 800/298; 435/6; 435/29; 435/41; 435/134; 435/189; 435/419; 435/243; 435/320.1; 536/23.1; 536/23.2; 554/230

(57) ABSTRACT

The present invention relates to enzymes which possess hydroxylase activity, in particular 42 hydroxylase activity, that can be used in methods of synthesizing modified fatty acids.

### FIELD OF THE INVENTION

The present invention relates to enzymes which possess hydroxylase activity, in particular Δ2 hydroxylase activity, that can be used in methods of synthesizing modified fatty acids.

### BACKGROUND OF THE INVENTION

Fatty acids are carboxylic acids with long-chain hydrocarbon side groups and play a fundamental role in many biological processes. Fatty acids are often unhydroxylated; however, such unhydroxylated fatty acids may be converted to hydroxyl fatty acids by the introduction of at least one hydroxyl group, a process catalyzed by a hydroxylase enzyme.

Hydroxyl fatty acids and hydroxyl oils are particularly important for a variety of industrial applications. For example, hydroxyl fatty acids, such as ricinoleic acid (12-hydroxyoctadec-9-enoic acid), are important industrial feedstock in the manufacture of biolubricants, functional fluids, ink, paints, coatings, nylons, resins, foams and other biopolymers.

The biosynthesis of fatty acids is a major activity of plants and microorganisms. Biotechnology has long been considered an efficient way to manipulate the process of producing fatty acids in plants and microorganisms. It is cost-effective and renewable with little side effects. Thus, industrial effort directed to the production of various compounds including speciality fatty acids and pharmaceutical polypeptides through the manipulation of plant, animal, and yeast cells has ensued.

At present, castor bean (Ricinus communis) is a major source for hydroxyl fatty acids. Due to poor agronomic performance and the presence of highly potent toxins and allergens in the seed, castor bean is not an ideal source for the fatty acids. Thus, a growing demand exists for alternatives to replace castor bean as a source of the hydroxyl fatty acids. Genes involved in the biosynthesis of hydroxyl fatty acids such as ricinoleic and lesqueroleic acids have been isolated from plant castor bean (Ricinus communis) and Lesquerella fendleri (van de Loo et al., 1995; Broun et al., 1998). Both genes encode oleate 12-hydroxylase, which introduces a hydroxyl group at position 12 of oleic acid. However, the introduction of the castor bean oleate hydroxylase into tobacco, Arabidopsis thaliana resulted in low to intermediate levels of ricinoleic acid accumulation in seeds (van de Loo et al., 1995; Broun and Somerville, 1997; Smith et al., 2003).

There is a need for the identification of further hydroxylases that can be used to produce hydroxylated fatty acids.

### SUMMARY OF THE INVENTION

The present inventors have produced recombinant transgenic cells which are capable of hydroxlating fatty acids.

Thus, in a first aspect the present invention provides a eukaryotic cell comprising an exogenous nucleic acid encoding a polypeptide with fatty acid Δ2 hydroxylase activity.

Preferably, the hydroxylase has an efficiency of conversion of oleic acid to 2-hydroxyoleic acid of at least 1%, more preferably at least 10%, more preferably at least 25%, more preferably at least 50%.

Preferably, the hydroxylase, when expressed in a yeast cell, is capable of converting at least 1%, more preferably at least 10%, of oleic acid in the yeast cell to 2-hydroxyoleic acid.

Preferably, the hydroxylase has an efficiency of conversion of palmitic acid to 2-hydroxypalmitic acid of at least 1%, more preferably at least 5%.

Preferably, the hydroxylase, when expressed in a yeast cell, is capable of converting at least 1%, more preferably at least 5%, of palmitic acid in the yeast cell to 2-hydroxypalmitic acid.

Preferably, the hydroxylase has an efficiency of conversion of palmitoleic acid to 2-hydroxy palmitoleic acid of at least 1%, more preferably at least 10%.

Preferably, the hydroxylase, when expressed in a yeast cell, is capable of converting at least 1%, more preferably at least 10%, of palmitoleic acid in the yeast cell to 2-hydroxyl palmitoleic acid.

In an embodiment, the polypeptide comprises amino acids having a sequence as set forth in any one of SEQ ID NOs :1, 2 and 4 to 12, a biologically active fragment thereof, or an amino acid sequence which is at least 30% identical to any one or more of the sequences set forth in SEQ ID NOs: 1, 2 and 4 to 12.

In a further embodiment, the polypeptide comprises amino acids having a sequence as provided in SEQ ID NO:1 or 2, a biologically active fragment thereof, or an amino acid sequence which is at least 30% identical to SEQ ID NO:1 and/or 2. More preferably, the polypeptide comprises amino acids having a sequence as provided in SEQ ID NO:1, a biologically active fragment thereof, or an amino acid sequence which is at least 30% identical to SEQ ID NO:1.

In an embodiment, the polypeptide is capable of converting oleic acid to 2-hydroxyoleic acid, myristic acid to 2-hydroxymyristic acid, palmitic acid to 2-hydroxypalmitic acid, and/or palmitoleic acid to hydroxy palmitoleic acid.

In a further embodiment, the cell comprises an increased level of 2-hydroxy fatty acids relative to a corresponding eukaryotic cell lacking the exogenous nucleic acid.

In yet another embodiment, the cell comprises an increased level of 2OH-C14:0, 2OH-C16:0, 2OH-C16:1^{Δ9} and/or 2OH-C18:1^{Δ9} fatty acids relative to a corresponding eukaryotic cell lacking the exogenous nucleic acid.

In another embodiment, the cell further comprises an exogenous polynucleotide encoding a diacylglycerol acyltransferase (DGAT), glycerol-3-phosphate acyltransferase (GPAT), 1-acyl-glycerol-3-phosphate acyltransferase (LPAAT), acyl-CoA:lysophosphatidylcholine acyltransferase (LPCAT), phospholipase A₂ (PLA₂), phospholipase C (PLC), phospholipase D (PLD), CDP-choline diacylglycerol choline phosphotransferase (CPT), or phoshatidylcholine diacylglycerol acyltransferase (PDAT), or a combination of two or more thereof.

In another embodiment, the cell further comprises an exogenous polynucleotide encoding a desaturase and/or an elongase. In an embodiment, the desaturase is Δ12 desaturase.

In another embodiment, the cell further comprises an introduced mutation or an exogenous polynucleotide which down regulates the production and/or activity of an endogenous enzyme of the cell selected from DGAT, GPAT, LPAAT, LPCAT, PLA₂, PLC, PLD, CPT, PDAT, a desaturase, or an elongase or a combination of two or more thereof. In an embodiment, the desaturase is a Δ15 desaturase. In another embodiment, the elongase is an elongase which elongates a C18 fatty acid.

In another embodiment, the exogenous polynucleotide is selected from: an antisense polynucleotide, a sense polynucleotide, a catalytic polynucleotide, a microRNA, a polynucleotide which encodes a polypeptide which binds the endogenous enzyme and a double stranded RNA.

Preferably, the double stranded RNA (dsRNA) molecule comprising an oligonucleotide which comprises at least 19 contiguous nucleotides of a polynucleotide encoding the endogenous enzyme, wherein the portion of the molecule that is double stranded is at least 19 basepairs in length and comprises said oligonucleotide.

Preferably, the dsRNA molecule is expressed from a single promoter, wherein the strands of the double stranded portion are linked by a single stranded portion.

In an embodiment the exogenous polynucleotide down regulates the production and/or activity of an endogenous enzyme does not significantly effect the production and/or activity of an enzyme encoded by a transgene in the cell.

In a further embodiment, for one or more transgenic polypeptide produced by the cell, the level and/or activity of an orthologous endogenous polypeptide is down-regulated when compared to an isogenic non-trangenic cell. In this embodiment, it is preferred that an orthologous endogenous polypeptide is not a hydroxylase. Furthermore, in this embodiment it is preferred that the cell is a plant cell.

In another embodiment, the polypeptide can be isolated from a plant.

The cell can be any cell type, preferably a plant cell or a cell suitable for fermentation.

Preferably, the plant cell is from any Brassica sp., Gossypium hirsutum, Linum usitatissimum, Helianthus sp., Carthamus tinctorius, Glycine max, Zea mays or Arabidopsis thaliana. The plant cell may be from Crambe abyssinica, Camelina sativa, Cuphea sp, Vernonia galamensis, or tobacco (Nicotiana tabacum). Preferably, the Brassica species is Brassica napus, Brassica juncea, Brassica rapa, or Brassica carinata. More preferably, the plant cell is from Linum usitatissimum or Carthamus tinctorius. In an embodiment, the plant cell is not from Glycine max or Arabidopsis thaliana or both.

In another aspect, the present invention provides a method of producing a transgenic cell which is capable of hydroxylating fatty acids at the Δ2 position, the method comprising

i) introducing into a eukaryotic cell an exogenous polynucleotide encoding a fatty acid hydroxylase, and

ii) analysing the cell or a tissue, organ or organism comprising the cell, or progeny thereof, for ability to produce Δ2 hydroxylated fatty acids.

In an embodiment, the method further comprises selecting a transgenic cell in which:

i) the hydroxylase has an efficiency of conversion of oleic acid to 2-hydroxyoleic acid of at least 1%, or at least 10%, or at least 25%, or at least 50%,

ii) the hydroxylase has an efficiency of conversion of palmitic acid to 2-hydroxypalmitic acid of at least 1%, or at least 5%,

iii) the hydroxylase has an efficiency of conversion of palmitoleic acid to 2-hydroxy palmitoleic acid of at least 1%, or at least 10%.

The cell can be any cell, preferably a plant cell or a cell suitable for fermentation.

In an embodiment, the cell is a plant cell and the method further comprises generating a transgenic plant.

In a further aspect, the present invention provides a process for determining whether a nucleic acid molecule encodes a fatty acid Δ2 hydroxylase comprising:

i) obtaining a nucleic acid molecule operably linked to a promoter, the nucleic acid molecule encoding a polypeptide comprising amino acids having a sequence as set forth in any one of SEQ ID NOs:1, 2 and 4 to 12 or having at least 30% amino acid identity to at least one of SEQ ID NOs 1, 2 and 4 to 12,

ii) introducing the nucleic acid molecule into a cell or cell-free expression system in which the promoter is active,

iii) determining whether the level of fatty acid Δ2 hydroxylation is modified relative to the cell or cell-free expression system before introduction of the nucleic acid molecule, and

iv) optionally, selecting a nucleic acid molecule which when expressed increased levels of fatty acid Δ2 hydroxylation.

In another aspect, the present invention provides a process for identifying a nucleic acid molecule encoding a fatty acid Δ2 hydroxylase comprising:

i) obtaining a nucleic acid molecule operably linked to a promoter, the nucleic acid molecule encoding a polypeptide comprising amino acids having a sequence more closely related to SEQ ID NO:2 than to SEQ ID NO:3,

ii) introducing the nucleic acid molecule into a cell or cell-free expression system in which the promoter is active,

iii) determining whether the level of fatty acid Δ2 hydroxylation is modified relative to the cell or cell-free expression system before introduction of the nucleic acid molecule, and

iv) optionally, selecting a nucleic acid molecule which when expressed increased levels of fatty acid Δ2 hydroxylation.

In an embodiment, the hydroxylase is capable of hydroxylating a C14, C16 and/or C18 fatty acid.

In another embodiment, the polypeptide is a plant polypeptide or mutant thereof

In a preferred embodiment, the polypeptide comprises amino acids having a sequence more closely related to SEQ ID NO:1 than to SEQ ID NO:3.

In a further aspect, the present invention provides a substantially purified and/or recombinant polypeptide which, when expressed in a yeast cell, has an efficiency of conversion of oleic acid to 2-hydroxyoleic acid of at least 1%, or at least 10%, or at least 25%, or at least 50%, when compared to a corresponding yeast cell lacking the exogenous nucleic acid.

In yet another aspect, the present invention provides a substantially purified and/or recombinant polypeptide which, when expressed in a yeast cell, has an efficiency of conversion of palmitic acid to 2-hydroxypalmitic acid of at least 1% when compared to a corresponding yeast cell lacking the exogenous nucleic acid.

In another aspect, the present invention provides a substantially purified and/or recombinant polypeptide which, when expressed in a yeast cell, has an efficiency of conversion of palmitoleic acid to 2-hydroxy palmitoleic acid of at least 1% when compared to a corresponding yeast cell lacking the exogenous nucleic acid.

In another aspect, the present invention provides a substantially purified and/or recombinant polypeptide comprising amino acids having a sequence as provided in any one of SEQ ID NOs: 1, 2 and 4 to 12, a biologically active fragment thereof, or an amino acid sequence which is at least 30% identical to any one or more of SEQ ID NOs: 1, 2 and 4 to 12, wherein the polypeptide has fatty acid Δ2 hydroxylase activity.

In an embodiment, a polypeptide of the invention comprises amino acids having a sequence as provided in SEQ ID NO:1 or 2, a biologically active fragment thereof, or an amino acid sequence which is at least 30% identical to SEQ ID NO:1 and/or 2. More preferably, the polypeptide comprises amino acids having a sequence as provided in SEQ ID NO:1, a biologically active fragment thereof, or an amino acid sequence which is at least 30% identical to SEQ ID NO:1.

In an embodiment, the fatty acid is a C16 or C18 fatty acid.

In a further embodiment, the polypeptide is a fusion protein further comprising at least one other polypeptide sequence. The at least one other polypeptide may be a polypeptide that enhances the stability of a polypeptide of the present invention, or a polypeptide that assists in the purification of the fusion protein.

In another aspect, the present invention provides an isolated and/or exogenous polynucleotide comprising:

i) a sequence of nucleotides selected from any one of SEQ ID NOs: 13 to 24,

ii) a sequence of nucleotides encoding a polypeptide of the invention,

iii) a sequence of nucleotides which are at least 50% identical to one or more of the sequences set forth in SEQ ID NOs:13 to 24, and/or

iv) a sequence which hybridises to any one of i) to iii) under stringent conditions.

In an embodiment, a polynucleotide of the invention comprises a sequence of nucleotides as provided in SEQ ID NO:13 or 14, or a nucleotide sequence which is at least 30% identical to SEQ ID NO:13 and/or 14. More preferably, a polynucleotide of the invention comprises a sequence of nucleotides as provided in SEQ ID NO:13, or a nucleotide sequence which is at least 30% identical to SEQ ID NO:13.

In another aspect, provided is chimeric vector comprising the polynucleotide of the invention.

Preferably, the polynucleotide is operably linked to a promoter.

Also provided is a cell comprising the recombinant polypeptide of the invention, the exogenous polynucleotide of the invention and/or the vector of the invention.

In an embodiment, the cell is a plant, fungal, yeast, bacterial or animal cell, more preferably a plant or fungal cell.

In another aspect, the present invention provides a method of producing the polypeptide of the invention, the method comprising expressing in a cell or cell free expression system the vector of the invention.

In an embodiment, the method further comprises isolating the polypeptide.

In yet a further aspect, the present invention provides a transgenic non-human organism comprising a cell of the invention.

Preferably, the organism is a transgenic plant.

Also provided is a seed comprising the cell of the invention.\

In a further aspect, the invention provides a method of producing seed, the method comprising,

a) growing a plant of the invention, and

b) harvesting the seed.

In a further aspect, the present invention provides oil, or fatty acid, produced by, or obtained from, the cell of the invention, the transgenic non-human organism of the invention, or the seed of the invention.

In an embodiment, the oil comprises 2OH-C14:0, 2OH-C16:0, 2OH-C16:1^{Δ9} and/or 2OH-C18:1^{Δ9}.

In a further embodiment, the oil is obtained by extraction of oil from an oilseed.

In another aspect, the present invention provides an oil comprising fatty acids comprising at least 1.0% (w/w) 2-hydroxyoleic acid as a percentage of the total fatty acid of the oil. In an embodiment, the oil comprises at least 3.0% (w/w) 2-hydroxyoleic acid as a percentage of the total fatty acid of the oil.

In a further aspect, the present invention provides an oil comprising fatty acids comprising at least 1.0% (w/w) 2-hydroxypalmitic acid as a percentage of the total fatty acid of the oil. In an embodiment, the oil comprises at least 3.0% (w/w) 2-hydroxypalmitic acid as a percentage of the total fatty acid of the oil.

In a further aspect, the present invention provides an oil comprising fatty acids comprising at least 0.5% (w/w) 2-hydroxymyristic acid as a percentage of the total fatty acid of the oil.

In a further aspect, the present invention provides an oil comprising fatty acids comprising at least 1.0% (w/w) 2-hydroxy palmitoleic acid as a percentage of the total fatty acid of the oil. In an embodiment, the oil comprises at least 3.0% (w/w) 2-hydroxy palmitoleic acid as a percentage of the total fatty acid of the oil.

In another aspect, the present invention provides a method of producing oil containing Δ2 hydroxylated fatty acids, the method comprising extracting oil from the cell of the invention, the transgenic non-human organism of the invention, or the seed of the invention, wherein at least 1% of the fatty acid content of the oil comprises a 2-hydroxy.

In an embodiment, the cell is of an organism suitable for fermentation and the method further comprises exposing the cell to at least one fatty acid precursor.

In another aspect, the present invention provides a fermentation process comprising the steps of:

i) providing a vessel containing a liquid composition comprising a cell of the invention, or an organism comprising said cell, which is suitable for fermentation, and constituents required for fermentation and fatty acid biosynthesis, and

ii) providing conditions conducive to the fermentation of the liquid composition contained in said vessel.

In another aspect, the present invention provides a method of performing a Δ2 hydroxylase reaction, the method comprising contacting a substrate saturated, monounsaturated or polyunsaturated fatty acid with the polypeptide of the invention.

In another aspect, the present invention provides a substantially purified antibody, or fragment thereof, that specifically binds a polypeptide of the invention.

In another aspect, the present invention provides an extract from the seed of the invention, the plant of the invention, the cell of the invention, and/or the transgenic non-human organism of the invention, wherein said extract comprises an increased level of Δ2 hydroxylated fatty acids relative to a corresponding extract from an isogenic non-transgenic seed, plant, cell or transgenic non-human organism.

In another aspect, the present invention provides for the use of a cell of the invention, the polypeptide of the invention, the polynucleotide of the invention, the vector of the invention, the transgenic non-human organism of the invention, the seed of the invention, oil of the invention, the fatty acid of the invention and/or the extract of the invention for the manufacture of an industrial product.

In another aspect, the present invention provides a composition comprising a cell of the invention, the polypeptide of the invention, the polynucleotide of the invention, the vector of the invention, the transgenic non-human organism of the invention, oil of the invention, the fatty acid of the invention, the extract of the invention and/or an antibody of the invention, and a suitable carrier.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1. GC-MS of yeast cell FAME-TMS derivatives expressing pYES2 vector only (A) or pYES2-AtFAH2 (B).

FIG. 2. Growth profile of S. cerevisiae YPH499 containing pYES₂::208870 (FAH2). Relative cell concentration was determined by measuring the optical density at 600 nm of diluted samples periodically removed from the culture.

FIG. 3. Phylogentic tree of FAH-related proteins.

FIG. 4. GC-MS of yeast cell FAME-TMS derivatives expressing pYES-DEST52 vector only (A) or pYES-LtFAH2 (B).

FIG. 5. Chemical structure of hexadeca-7,9-diynoic acid and hexadeca-10,12-diynoic acid.

FIG. 6. Chemical structure of 2-hydroxy-hexadeca-7,9-diynoic acid and 2-hydroxy-hexadeca-10,12-diynoic acid.

### KEY TO THE SEQUENCE LISTING

SEQ ID NO:1—Amino acid sequence of Thymus citriodorus fatty acid hydroxylase (Thyci FAH).

SEQ ID NO:2—Amino acid sequence of Arabidopsis thaliana fatty acid hydroxylase 2 (Arath FAH2).

SEQ ID NO:3—Amino acid sequence of Arabidopsis thaliana fatty acid hydroxylase 1 (Arath FAH1).

SEQ ID NO:4—Amino acid sequence of Brassica rapa fatty acid hydroxylase 2 (Brara FAH2).

SEQ ID NO:5—Amino acid sequence of Glycine max fatty acid hydroxylase 1 (Glyma FAH1).

SEQ ID NO:6—Amino acid sequence of Glycine max fatty acid hydroxylase 2a (Glyma FAH2a).

SEQ ID NO:7—Amino acid sequence of Lycopersicon esculentum fatty acid hydroxylase 1 (Lyces FAH1).

SEQ ID NO:8—Amino acid sequence of Solanum tuberosum fatty acid hydroxylase (Soltu FAH).

SEQ ID NO:9—Amino acid sequence of Ipomoea nil fatty acid hydroxylase 2 (Ipo FAH2).

SEQ ID NO:10—Amino acid sequence of Nicotiana tabacum fatty acid hydroxylase 2 (Nicta FAH2). SEQ ID NO:11—Amino acid sequence of Oryza sativa fatty acid hydroxylase 2 (Orysa FAH2).

SEQ ID NO:12—Amino acid sequence of Triticum aestivum fatty acid hydroxylase 2 (Triae FAH2).

SEQ ID NO:13—Coding sequence for Thymus citriodorus fatty acid hydroxylase (Thyci FAH).

SEQ ID NO:14—Coding sequence for Arabidopsis thaliana fatty acid hydroxylase 2 (Arath FAH2).

SEQ ID NO:15—Coding sequence for Arabidopsis thaliana fatty acid hydroxylase 1 (Arath FAH1).

SEQ ID NO:16—Coding sequence for Brassica rapa fatty acid hydroxylase 2 (Brara FAH2).

SEQ ID NO:17—Coding sequence for Glycine max fatty acid hydroxylase 1 (Glyma FAH1).

SEQ ID NO:18—Coding sequence for Glycine max fatty acid hydroxylase 2a (Glyma FAH2a).

SEQ ID NO:19—Coding sequence for Lycopersicon esculentum fatty acid hydroxylase 1 (Lyces FAH1).

SEQ ID NO:20—Coding sequence for Solanum tuberosum fatty acid hydroxylase (Soltu FAH).

SEQ ID NO:21—Coding sequence for Ipomoea nil fatty acid hydroxylase 2 (Ipo FAH2).

SEQ ID NO:22—Coding sequence for Nicotiana tabacum fatty acid hydroxylase 2 (Nicta FAH2).

SEQ ID NO:23—Coding sequence for Oryza sativa fatty acid hydroxylase 2 (Orysa FAH2).

SEQ ID NO:24—Coding sequence for Triticum aestivum fatty acid hydroxylase 2 (Triae FAH2).

SEQ ID NO's 25 to 39—Oligonucleotide primers.

SEQ ID NO:40—Amino acid sequence of motif 1 in Arath FAH1.

SEQ ID NO:41—Amino acid sequence of motif 2 in Arath FAH1.

SEQ ID NO:42—Amino acid sequence of motif 3 in Arath FAH1.

SEQ ID NO:43—Amino acid sequence of motif 4 in Arath FAH1.

SEQ ID NO:44—Amino acid sequence of motif 5 in Arath FAH1.

SEQ ID NO:45—Amino acid sequence of motif 6 in Arath FAH1.

SEQ ID NO:46—Amino acid sequence of motif 7 in Arath FAH1.

SEQ ID NO:47—Amino acid sequence of motif 8 in Arath FAH1.

SEQ ID NO:48—Amino acid sequence of motif 9 in Arath FAH1.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, fatty acid chemistry and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D.M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

### Selected Definitions

As used herein, the term “oil” refers to a composition which comprises at least 60% (w/w) lipid. Oil is typically a liquid at room temperature. Preferably, the lipid predominantly comprises fatty acids that are at least 16 carbons in length. The fatty acids are typically in an esterified form, such as for example as triacylglycerols, acyl-CoA or phospholipid. The fatty acids may be free fatty acids and/or be found as triacylglycerols (TAGs). In an embodiment, at least 50%, more preferably at least 70%, more preferably at least 80% of the fatty acids in seedoil of the invention can be found as TAGs. “Oil” of the invention may be “seedoil” if it is obtained from seed. Oil may be present in or obtained from cells, tissues, organs or organisms other than seeds, in which case the oil is not seedoil as defined herein.

As used herein, the term “seedoil” refers to a composition obtained from the seed/grain of a plant which comprises at least 60% (w/w) lipid. Seedoil is typically a liquid at room temperature. Preferably, the lipid predominantly (>50%) comprises fatty acids that are at least 16 carbons in length. More preferably, at least 50% of the total fatty acids in the seedoil are C18 fatty acids. The fatty acids are typically in an esterified form, such as for example as triacylglycerols, acyl-CoA or phospholipid. The fatty acids may be free fatty acids and/or be found esterified such as triacylglycerols (TAGs). In an embodiment, at least 50%, more preferably at least 70%, more preferably at least 80% or at least 90% of the fatty acids in seedoil of the invention can be found as TAGs. Seedoil of the invention can form part of the grain/seed or portion thereof. Alternatively, seedoil of the invention has been extracted from grain/seed. Thus, in an embodiment, “seedoil” of the invention is “substantially purified” or “purified” oil that has been separated from one or more other lipids, nucleic acids, polypeptides, or other contaminating molecules with which it is associated in its native state. It is preferred that the substantially purified oil is at least 60% free, more preferably at least 75% free, and more preferably at least 90% free from other components with which it is naturally associated. Seedoil of the invention may further comprise non-fatty acid molecules such as, but not limited to, sterols. In an embodiment, the seedoil is canola oil (Brassica napus, Brassica rapa ssp.), mustard oil (Brassica juncea), other Brassica oil, sunflower oil (Helianthus annus), linseed oil (Linum usitatissimum), soybean oil (Glycine max), safflower oil (Carthamus tinctorius), corn oil (Zea mays), tobacco oil (Nicotiana tabacum), peanut oil (Arachis hypogaea), palm oil, cottonseed oil (Gossypium hirsutum), coconut oil (Cocos nucifera), avocado oil (Persea americana), olive oil (Olea europaea), cashew oil (Anacardium occidentale), macadamia oil (Macadamia intergrifolia), almond oil (Prunus amygdalus) or Arabidopsis seed oil (Arabidopsis thaliana). Seedoil may be extracted from seed by any method known in the art. This typically involves extraction with nonpolar solvents such as diethyl ether, petroleum ether, chloroform/methanol or butanol mixtures. Lipids associated with the starch in the grain may be extracted with water-saturated butanol. The seedoil may be “de-gummed” by methods known in the art to remove polysaccharides or treated in other ways to remove contaminants or improve purity, stability or colour. The triacylglycerols and other esters in the oil may be hydrolysed to release free fatty acids, or the oil hydrogenated or treated chemically or enzymatically as known in the art.

As used herein, the term “fatty acid” refers to a carboxylic acid (or organic acid), often with a long aliphatic tail, either saturated or unsaturated. Typically fatty acids have a carbon-carbon bonded chain of at least 8 carbon atoms in length, more preferably at least 12 carbons in length. Most naturally occurring fatty acids have an even number of carbon atoms because their biosynthesis involves acetate which has two carbon atoms. The fatty acids may be in a free state (non-esterified) or in an esterified form such as part of a triglyceride, diacylglyceride, monoacylglyceride, acyl-CoA (thio-ester) bound or other bound form. The fatty acid may be esterified as a phospholipid such as a phosphatidylcholine, phosphatidylethanolamine, phosphatidylserine, phosphatidylglycerol, phosphatidylinositol or diphosphatidylglycerol forms. The terms “fatty acid” and “fatty acids” are generally used interchangeably, however, as the skilled person will appreciate seedoil will comprise more than a single fatty acid molecule and generally more than one type of fatty acid.

Triacylglyceride (TAG) is glyceride in which the glycerol is esterified with three fatty acids. In the Kennedy pathway of TAG synthesis, the precursor sn-glycerol-3-phosphate is esterified by a fatty acid coenzyme A ester in a reaction catalysed by a glycerol-3-phosphate acyltransferase at position sn-1 to form lysophosphatidic acid (LPA), and this is in turn acylated by an acylglycerophosphate acyltransferase in position sn-2 to form phosphatidic acid. The phosphate group is removed by the enzyme phosphatidic phosphohydrolase, and the resultant 1,2-diacyl-sn-glycerol (DAG) is acylated by a diacylglycerol acyltransferase to form the triacyl-sn-glycerol.

“Saturated fatty acids” do not contain any double bonds or other functional groups along the chain. The term “saturated” refers to hydrogen, in that all carbons (apart from the carboxylic acid [—COOH] group) contain as many hydrogens as possible. In other words, the omega (ω) end contains 3 hydrogens (CH3-) and each carbon within the chain contains 2 hydrogens (—CH2-).

“Unsaturated fatty acids” are of similar form to saturated fatty acids, except that one or more alkene functional groups exist along the chain, with each alkene substituting a singly-bonded ”—CH2-CH2-” part of the chain with a doubly-bonded”—CH═CH—” portion (that is, a carbon double bonded to another carbon). The two next carbon atoms in the chain that are bound to either side of the double bond can occur in a cis or trans configuration.

As used herein, the terms “monounsaturated fatty acid” refers to a fatty acid which comprises at least 12 carbon atoms in its carbon chain and only one alkene group in the chain. As used herein, the terms “polyunsaturated fatty acid” or “PUFA” refer to a fatty acid which comprises at least 12 carbon atoms in its carbon chain and at least two alkene groups (carbon-carbon double bonds). Ordinarily, the number of carbon atoms in the carbon chain of the fatty acids refers to an unbranched carbon chain. If the carbon chain is branched, the number of carbon atoms excludes those in sidegroups. In one embodiment, the long-chain polyunsaturated fatty acid is an ω3 fatty acid, that is, having a desaturation (carbon-carbon double bond) in the third carbon-carbon bond from the methyl end of the fatty acid. In another embodiment, the long-chain polyunsaturated fatty acid is an ω6 fatty acid, that is, having a desaturation (carbon-carbon double bond) in the sixth carbon-carbon bond from the methyl end of the fatty acid.

As used herein, the terms “long-chain polyunsaturated fatty acid” or “LC-PUPA” refer to a fatty acid which comprises at least 20 carbon atoms in its carbon chain and at least two carbon-carbon double bonds.

“Δ2 hydroxylase” or “fatty acid Δ2 hydroxylase” and variations thereof as used herein, refers to an enzyme that introduces a hydroxyl group into a fatty acid at the Δ2 position resulting at least in the production of a Δ2 hydroxylated fatty acid. An example of a Δ2 hydroxylase provided herein comprises an amino acid sequence as provided in SEQ ID NO:1. Other Δ2 hydroxylases can be identified by homology to SEQ ID NO:1, for example SEQ ID Nos: 4 to 12. Enzymatic activity can readily be tested in recombinant cells such as yeast cells as described herein.

The level of production of Δ2 hydroxylated fatty acids in a recombinant cell may also be expressed as a conversion ratio, i.e. the amount of the Δ2 hydroxylated fatty acids formed as a percentage of one or more substrate fatty acids. This is referred to herein as the “efficiency of conversion”. In a preferred embodiment, the “efficiency of conversion” is determined by analysing a recombinant yeast cell as described in Example 2.

As used herein, the term “diacylglycerol acyltransferase” (EC 2.3.1.20; DGAT) refers to a protein which transfers a fatty acyl group from acyl-CoA to a diacylglycerol substrate to produce a triacylglycerol. Thus, the term “diacylglycerol acyltransferase activity” refers to the transfer of acyl-CoA to diacylglycerol to produce triacylglycerol. There are three known types of DGAT referred to as DGAT1, DGAT2 and DGAT3 respectively. DGAT1 polypeptides typically have 10 transmembrane domains, DGAT2 typically have 2 transmembrane domains, whilst DGAT3 is typically soluble. Examples of DGAT1 polypeptides include proteins encoded by DGAT1 genes from Aspergillus fumigatus (Accession No. XP_{—}755172), Arabidopsis thaliana (CAB44774), Ricinus communis (AAR11479), Vernicia fordii (ABC94472), Vernonia galamensis (ABV21945, ABV21946), Euonymus alatus (AAV31083), Caenorhabditis elegans (AAF82410), Rattus norvegicus (NP_{—}445889), Homo sapiens (NP_{—}036211), as well as variants and/or mutants thereof. Examples of DGAT2 polypeptides include proteins encoded by DGAT2 genes from Arabidopsis thaliana (Accession No. NP_{—}566952), Ricinus communis (AAY16324), Vernicia fordii (ABC94474), Mortierella ramanniana (AAK84179), Homo sapiens (Q96PD7, Q58HT5), Bos taurus (Q70VD8), Mus musculus (AAK84175), as well as variants and/or mutants thereof. Examples of DGAT3 polypeptides include proteins encoded by DGAT3 genes from peanut (Arachis hypogaea, Saha, et al., 2006), as well as variants and/or mutants thereof.

As used herein, the term “phospholipase A₂” (PLA₂) refers to a protein which hydrolyzes the sn2-acyl bond of phospholipids to produce arachidonic acid and lysophospholipids. Thus, the term “phospholipase A₂ activity” refers to the hydrolysis of the sn2-acyl bond of phospholipids to produce arachidonic acid and lysophospholipids. Examples of phospholipase A₂ polypeptides include proteins encoded by PLA₂ genes from Arabidopsis such as -a (At2g06925, AY136317), AtsPLA₂-β (At2g19690, AY136317), AtsPLA₂-γ (At4g29460, AY148346), AtsPLA₂-δ (At4g29470, AY148347) and PLA₂s (At3g45880, AK226677 and At1g61850, NM_{—}104867), as well as variants and/or mutants thereof.

As used herein, the term “phosphatidylcholine diacylglycerol acyltransferase” (PDAT) refers to a protein which transfers an acyl group from phosphatidylcholine to diacylglycerol. Thus, the term “phosphatidylcholine diacylglycerol acyltransferase activity” refers to the transfer of phosphatidylcholine onto diacylglycerol to produce triacylglycerol.

As used herein, the term “CDP-choline diacylglycerol choline phosphotransferase” (CPT), refers to a protein which reversibly transfers phosphatidylcholine groups onto diacylglycerol. Thus, the term “CDP-choline diacylglycerol choline phosphotransferase activity” refers to the reversible transfer of phosphatidylcholine groups onto diacylglycerol.

As used herein, the term “acyl-CoA:lysophosphatidylcholine acyltransferase” (LPCAT) refers to a protein which reversibly catalyzes the acyl-CoA-dependent acylation of lysophophatidylcholine to produce phosphatidylcholine and CoA. Thus, the term “acyl-CoA:lysophosphatidylcholine acyltransferase activity” refers to the reversible acylation of lysophophatidylcholine to produce phosphatidylcholine and CoA.

As used herein, the term “phospholipase C” (PLC) refers to a protein which hydrolyzes PIP₂ to produce diacylglycerol. Thus, the term “phospholipase C activity” refers to the hydrolysis of PIP₂ to produce diacylglycerol.

As used herein, the term “phospholipase D” (PLD) refers to a protein which hydrolyzes phosphatidylcholine to produce phosphatidic acid and a choline headgroup. Thus, the term “phospholipase D activity” refers to the hydrolysis of phosphatidylcholine to produce phosphatidic acid and a choline headgroup.

As used herein, the term “glycerol-3-phosphate acyltransferase” (GPAT) refers to a protein which acylates sn-glycerol-3-phosphate to form 1-acyl-sn-glycerol-3-phosphate. Thus, the term “glycerol-3-phosphate acyltransferase activity” refers to the acylation of sn-glycerol-3-phosphate to form 1-acyl-sn-glycerol-3-phosphate.

As used herein, the term “1-acyl-glycerol-3-phosphate acyltransferase” (LPAAT) refers to a protein which acylates sn-1-acyl-glycerol-3-phosphate at the sn-2 position to form phosphatidic acid. Thus, the term “1-acyl-glycerol-3-phosphate acyltransferase activity” refers to the acylation of sn-1-acyl-glycerol-3-phosphate at the sn-2 position to produce phosphatidic acid.

As used herein, a “desaturase”, “fatty acid desaturase” or variations thereof is an enzyme which removes two hydrogen atoms from the carbon chain of the fatty acid creating a carbon-carbon double bond. Desaturases are classified as; i) delta—indicating that the double bond is created at a fixed position from the carboxyl group of a fatty acid (for example, Δ12 desaturase creates a double bond at the 12th position from the carboxyl end), or ii) omega (e.g. ω3desaturase)—indicating the double bond is created at a specific position from the methyl end of the fatty acid. Examples of desaturases include those described in WO 2005/103253.

Biochemical evidence suggests that the fatty acid elongation consists of 4 steps: condensation, reduction, dehydration and a second reduction. In the context of this invention, an “elongase” refers to the polypeptide that catalyses the condensing step in the presence of the other members of the elongation complex, under suitable physiological conditions. It has been shown that heterologous or homologous expression in a cell of only the condensing component (“elongase”) of the elongation protein complex is required for the elongation of the respective acyl chain. Thus the introduced elongase is able to successfully recruit the reduction and dehydration activities from the transgenic host to carry out successful acyl elongations. The specificity of the elongation reaction with respect to chain length and the degree of desaturation of fatty acid substrates is thought to reside in the condensing component. This component is also thought to be rate limiting in the elongation reaction. Two groups of condensing enzymes have been identified so far. The first are involved in the extension of saturated and monounsaturated fatty acids (C18-22) such as, for example, the FAE1 gene of Arabidopsis. An example of a product formed is erucic acid (22:1) in Brassicas. This group are designated the FAE-like enzymes and do not appear to have a role in LC-PUFA biosynthesis. The other identified class of fatty acid elongases, designated the ELO family of elongases, are named after the ELO genes whose activities are required for the synthesis of the very long-chain fatty acids of sphingolipids in yeast. Apparent paralogs of the ELO-type elongases isolated from LC-PUPA synthesizing organisms like algae, mosses, fungi and nematodes have been shown to be involved in the elongation and synthesis of LC-PUFA. Examples of elongases include those described in WO 2005/103253.

As used herein, the term “an exogenous polynucleotide which down regulates the production and/or activity of an endogenous enzyme” or variations thereof, refers to a polynucleotide that encodes an RNA molecules that down regulates the production and/or activity (for example, encoding an siRNA), or the exogenous polynucleotide itself down regulates the production and/or activity (for example, an siRNA is delivered to directly to, for instance, a cell).

As used herein, the phrase “does not significantly effect the production and/or activity of an enzyme encoded by a transgene” means that the level of activity of the enzyme is at least 75%, more preferably at least 90%, of the level of an isogenic transgenic cell lacking the exogenous polynucleotide that down regulates the production and/or activity of an endogenous enzyme.

As used herein, the term “more closely related” refers to the primary amino acid sequence relationship of the relevant polypeptides. This can be determined using standard techniques in the art such as those described herein relating to the determination of % identity.

The term “plant” includes whole plants, vegetative structures (for example, leaves, stems), roots, floral organs/structures, seed (including embryo, endosperm, and seed coat), plant tissue (for example, vascular tissue, ground tissue, and the like), cells and progeny of the same. The plant, seed, plant part or plant cells may be, or from, monocotyledonous plants or preferably dicotyledonous plants.

A “transgenic cell”, “genetically modified cell”, “recombinant cell” or variations thereof refers to a cell that contains a gene construct (“transgene”) not found in a wild-type cell of the same species, variety or cultivar.

A “transgenic seed”, “genetically modified seed”, “recombinant seed” or variations thereof refers to a seed that contains a gene construct (“transgene”) not found in a wild-type seed from the same species, variety or cultivar of plant.

A “transgenic plant”, “genetically modified plant” “recombinant plant” or variations thereof refers to a plant that contains a gene construct (“transgene”) not found in a wild-type plant of the same species, variety or cultivar.

A “transgene” as referred to herein has the normal meaning in the art of biotechnology and includes a genetic sequence which has been produced or altered by recombinant DNA or RNA technology and which has been introduced into the plant or other cell. The transgene may include genetic sequences derived from a plant cell. Typically, the transgene has been introduced into the plant or other cell by human manipulation such as, for example, by transformation but any method can be used as one of skill in the art recognizes.

“Grain” as used herein generally refers to mature, harvested grain but can also refer to grain after imbibition or germination, according to the context. Mature grain commonly has a moisture content of less than about 18-20%. “Seed” as used herein includes mature seed such as is typically harvested from a plant and developing seed as is typically found in a plant during growth. Mature seed is typically dormant i.e. in a resting state.

As used herein, the term “wild-type” or variations thereof refers to a cell, tissue, seed or plant that has not been modified according to the invention. “Isogenic” refers to a cell, tissue, seed or plant which differs from a reference cell, tissue, seed or plant at one or more, generally not more than a few such as two, three or four, genetic loci, resulting in an alteration of one or more traits. The genetic loci(us) may have a single gene or genetic construct, or multiple genes or genetic constructs (generally not more than a few such as two, three or four), typically a transgene(s). A “corresponding” cell, tissue, seed or plant as used herein refers to a second cell, tissue, seed or plant which lacks the gene(s) or constructs, which differs from the first cell, tissue, seed or plant essentially by only that gene(s) or construct(s), and which typically has been treated in the same manner e.g. temperature, culture conditions etc, as the first. Isogenic wildtype cells, tissue or plants may be used as controls to compare levels of expression of an exogenous nucleic acid or the extent and nature of trait modification with cells, tissue or plants modified as described herein.

“Operably linked” as used herein refers to a functional relationship between two or more nucleic acid (e.g., DNA) segments. Typically, it refers to the functional relationship of transcriptional regulatory element (promoter) to a transcribed sequence. For example, a promoter is operably linked to a coding sequence, such as a polynucleotide defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

As used herein, the term “gene” is to be taken in its broadest context and includes the deoxyribonucleotide sequences comprising the protein coding region of a structural gene and including sequences located adjacent to the coding region on both the 5′ and 3′ ends for a distance of at least about 2 kb on either end and which are involved in expression of the gene. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region which may be interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

As used herein, the term “can be isolated from” means that the polynucleotide or encoded polypeptide is naturally produced by an organism, particularly an insect.

The term “extract” refers to any part of the cell or organism such as a plant. An “extract” typically involves the disruption of cells and possibly the partial purification of the resulting material. Naturally, the “extract” will comprise at least one hydroxylated fatty acid. Extracts can be prepared using standard techniques of the art.

### Cells

Suitable cells of the invention include any cell that can be transformed with a polynucleotide encoding a polypeptide/enzyme described herein, and which is thereby capable of being used for producing hydroxylated fatty acids. Host cells into which the polynucleotide(s) are introduced can be either untransformed cells or cells that are already transformed with at least one nucleic acid molecule. Such nucleic acid molecule may related to fatty acid synthesis, TAG synthesis, or unrelated. Host cells of the present invention either can be endogenously (i.e., naturally) capable of producing proteins of the present invention or can be capable of producing such proteins only after being transformed with at least one nucleic acid molecule.

The cells may be prokaryotic or eukaryotic. Host cells of the present invention can be any cell capable of producing at least one protein described herein, and include bacterial, fungal (including yeast), parasite, arthropod, animal and plant cells. Preferred cells are eukaryotic cells, more preferred cells are yeast and plant cells. In a preferred embodiment, the plant cells are seed cells. The cells may be in cell culture. The cells may be isolated cells, or alternatively, cells that are or were part of a multicellular organism such as a plant or fungus. The cells may be comprised in a plant part such as a seed. The organism may be non-human.

In one particularly preferred embodiment, the cells may be of an organism suitable for fermentation. As used herein, the term the “fermentation process” refers to any fermentation process or any process comprising a fermentation step. A fermentation process includes, without limitation, fermentation processes used to produce alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, beta-carotene); and hormones. Fermentation processes also include fermentation processes used in the consumable alcohol industry (e.g., beer and wine), dairy industry (e.g., fermented dairy products), leather industry and tobacco industry. Preferred fermentation processes include alcohol fermentation processes, as are well known in the art. Preferred fermentation processes are anaerobic fermentation processes, as are well known in the art.

Suitable fermenting cells, typically microorganisms are able to ferment, i.e., convert, sugars, such as glucose or maltose, directly or indirectly into the desired fermentation product. Examples of fermenting microorganisms include fungal organisms, such as yeast. As used herein, “yeast” includes Saccharomyces spp.; Saccharomyces cerevisiae, Saccharomyces carlbergensis, Candida spp., Kluveromyces spp., Pichia spp., Hansenula spp., Trichoderma spp., Lipomyces starkey, and Yarrowia lipolytica. Preferred yeast include strains of the Saccharomyces spp., and in particular, Saccharomyces cerevisiae. Commercially available yeast include, e.g., Red Star/Lesaffre Ethanol Red (available from Red Star/Lesaffre, USA) FALI (available from Fleischmann's Yeast, a division of Burns Philp Food Inc., USA), SUPERSTART (available from Alltech), GERT STRAND (available from Gert Strand AB, Sweden) and FERMIOL (available from DSM Specialties).

In one embodiment, the cell is an animal cell or an algal cell. The animal cell may be of any type of animal such as, for example, a non-human animal cell, a non-human vertebrate cell, a non-human mammalian cell, or cells of aquatic animals such as fish or crustacea, invertebrates, insects, etc.

An example of a bacterial cell useful as a host cell of the present invention is Synechococcus spp. (also known as Synechocystis spp.), for example Synechococcus elongatus.

### Polypeptides

By “substantially purified polypeptide” or “purified polypeptide” we mean a polypeptide that has generally been separated from the lipids, nucleic acids, other peptides, and other contaminating molecules with which it is associated in its native state. Preferably, the substantially purified polypeptide is at least 60% free, more preferably at least 75% free, and more preferably at least 90% free from other components with which it is naturally associated.

The term “recombinant” in the context of a polypeptide refers to the polypeptide when produced by a cell, or in a cell-free expression system, in an altered amount or at an altered rate compared to its native state. In one embodiment the cell is a cell that does not naturally produce the polypeptide. However, the cell may be a cell which comprises a non-endogenous gene that causes an altered amount of the polypeptide to be produced. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components.

The terms “polypeptide” and “protein” are generally used interchangeably.

The % identity of a polypeptide is determined by GAP (Needleman and Wunsch, 1970) analysis (GCG program) with a gap creation penalty=5, and a gap extension penalty=0.3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. More preferably, the query, sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns two sequences over their entire length.

As used herein a “biologically active” fragment is a portion of a polypeptide of the invention which maintains a defined activity of the full-length polypeptide, namely possessing Δ2 hydroxylase activity. Biologically active fragments can be any size as long as they maintain the defined activity. Preferably, the biologically active fragment maintains at least 10% of the activity of the full length protein.

With regard to a defined polypeptide/enzyme, it will be appreciated that % identity figures higher than those provided above will encompass preferred embodiments. Thus, where applicable, in light of the minimum % identity figures, it is preferred that the polypeptide/enzyme comprises an amino acid sequence which is at least 60%, more preferably at least 65%, more preferably at least 70%, more preferably at least 75%, more preferably at least 76%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final peptide product possesses the desired characteristics. Preferred amino acid sequence mutants have only one, two, three, four or less than 10 amino acid changes relative to the reference wildtype polypeptide.

Mutant (altered) peptides can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). Products derived from mutated/altered DNA can readily be screened using techniques described herein to determine if they possess Δ2 hydroxylase activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as the active site(s). Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

In a preferred embodiment a mutant/variant polypeptide has one or two or three or four conservative amino acid changes when compared to a naturally occurring polypeptide. Details of conservative amino acid changes are provided in Table 1. In a preferred embodiment, the changes are not in one or more of the motifs which are highly conserved between the different hydroxylases provided herewith. As the skilled person would be aware, such minor changes can reasonably be predicted not to alter the activity of the polypeptide when expressed in a recombinant cell.

Furthermore, if desired, unnatural amino acids or chemical amino acid analogues can be introduced as a substitution or addition into the polypeptides of the present invention. Such amino acids include, but are not limited to, the D-isomers of the common amino acids, 2,4-diaminobutyric acid, a-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

Polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Polynucleotides

By “isolated polynucleotide” we mean a polynucleotide which has generally been separated from the polynucleotide sequences with which it is associated or linked in its native state. Preferably, the isolated polynucleotide is at least 60% free, more preferably at least 75% free, and more preferably at least 90% free from other components with which it is naturally associated. Furthermore, the term “polynucleotide” is used interchangeably herein with the terms “nucleic acid molecule”, “gene” and “mRNA”.

The term “exogenous” in the context of a polynucleotide (nucleic acid) refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically such chimeric polynucleotides comprise at least an open reading frame encoding a polypeptide of the invention operably linked to a promoter suitable of driving transcription of the open reading frame in a cell of interest.

“Polynucleotide” refers to a oligonucleotide, polynucleotide or any fragment thereof. It may be DNA or RNA of genomic or synthetic origin, double-stranded or single-stranded, and combined with carbohydrate, lipids, protein, or other materials to perform a particular activity defined herein.

The % identity of a polynucleotide is determined by GAP (Needleman and Wunsch, 1970) analysis (GCG program) with a gap creation penalty=5, and a gap extension penalty=0.3. The query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. Even more preferably, the query sequence is at least 300 nucleotides in length and the GAP analysis aligns the two sequences over a region of at least 300 nucleotides. Even more preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In a further embodiment, the present invention relates to polynucleotides which are substantially identical to those specifically described herein. As used herein, with reference to a polynucleotide the term “substantially identical” means the substitution of one or a few (for example 2, 3, or 4) nucleotides whilst maintaining at least one activity of the native protein encoded by the polynucleotide. In addition, this term includes the addition or deletion of nucleotides which results in the increase or decrease in size of the encoded native protein by one or a few (for example 2, 3, or 4) amino acids whilst maintaining at least one activity of the native protein encoded by the polynucleotide.

Oligonucleotides of the present invention can be RNA, DNA, or derivatives of either. The minimum size of such oligonucleotides is the size required for the formation of a stable hybrid between an oligonucleotide and a complementary sequence on a nucleic acid molecule of the present invention. Preferably, the oligonucleotides are at least 15 nucleotides, more preferably at least 18 nucleotides, more preferably at least 19 nucleotides, more preferably at least 20 nucleotides, even more preferably at least 25 nucleotides in length. The present invention includes oligonucleotides that can be used as, for example, probes to identify nucleic acid molecules, or primers to produce nucleic acid molecules. Oligonucleotide of the present invention used as a probe are typically conjugated with a label such as a radioisotope, an enzyme, biotin, a fluorescent molecule or a chemiluminescent molecule.

Probes and/or primers can be used to clone homologues of the polynucleotides of the invention from other species. Furthermore, hybridization techniques known in the art can also be used to screen genomic or cDNA libraries for such homologues.

Polynucleotides and oligonucleotides of the present invention include those which hybridize under stringent conditions to a sequence provided as SEQ ID NO's: 13 to 24. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

Usually, monomers of a polynucleotide or oligonucleotide are linked by phosphodiester bonds or analogs thereof to form oligonucleotides ranging in size from a relatively short monomeric units, e.g., 12-18, to several hundreds of monomeric units. Analogs of phosphodiester linkages include: phosphorothioate, phosphorodithioate, phosphoroselenoate, phosphorodiselenoate, phosphoroanilothioate, phosphoranilidate, phosphoramidate.

### Antisense Polynucleotides

The term “antisense polynucleotide” shall be taken to mean a DNA or RNA, or combination thereof, molecule that is complementary to at least a portion of a specific mRNA molecule encoding a polypeptide defined herein and capable of interfering with a post-transcriptional event such as mRNA translation. The use of antisense methods is well known in the art (see for example, G. Hartmann and S. Endres, Manual of Antisense Methodology, Kluwer (1999)). The use of antisense techniques in plants has been reviewed by Bourque, 1995 and Senior, 1998. Bourque, 1995 lists a large number of examples of how antisense sequences have been utilized in plant systems as a method of gene inactivation. She also states that attaining 100% inhibition of any enzyme activity may not be necessary as partial inhibition will more than likely result in measurable change in the system. Senior (1998) states that antisense methods are now a very well established technique for manipulating gene expression.

An antisense polynucleotide of the invention will hybridize to a target polynucleotide under physiological conditions. As used herein, the term “an antisense polynucleotide which hybridises under physiological conditions” means that the polynucleotide (which is fully or partially single stranded) is at least capable of forming a double stranded polynucleotide with mRNA encoding a protein under normal conditions in a cell, preferably a plant cell.

Antisense molecules may include sequences that correspond to the structural genes or for sequences that effect control over the gene expression or splicing event. For example, the antisense sequence may correspond to the targeted coding region of the genes of the invention, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

### Catalytic Polynucleotides

The term catalytic polynucleotide/nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Haseloff and Gerlach, 1988; Perriman et al., 1992) and the hairpin ribozyme (Shippy et al., 1999).

The ribozymes of this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. Accordingly, also provided by this invention is a nucleic acid molecule, i.e., DNA or cDNA, coding for a catalytic polynucleotide of the invention. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase.

As with antisense polynucleotides described herein, catalytic polynucleotides of the invention should also be capable of hybridizing a target nucleic acid molecule under “physiological conditions”, namely those conditions within a cell (especially conditions in a plant cell).

### RNA Interference

The terms “RNA interference”, “RNAi” or “gene silencing” refers generally to a process in which a double-stranded RNA molecule reduces the expression of a nucleic acid sequence with which the double-stranded RNA molecule shares substantial or total homology. However, it has more recently been shown that RNA interference can be achieved using non-RNA double stranded molecules (see, for example, US 20070004667).

RNA interference (RNAi) is particularly useful for specifically inhibiting the production of a particular protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof, in this case an mRNA encoding a polypeptide according to the invention. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Waterhouse et al. (1998), Smith et al. (2000), WO 99/32619, WO 99/53050, WO 99/49029, and WO 01/34815.

In one example, a DNA is introduced that directs the synthesis of an at least partly double stranded RNA product(s) with homology to the target gene to be inactivated. The DNA therefore comprises both sense and antisense sequences that, when transcribed into RNA, can hybridize to form the double-stranded RNA region. In a preferred embodiment, the sense and antisense sequences are separated by a spacer region that comprises an intron which, when transcribed into RNA, is spliced out. This arrangement has been shown to result in a higher efficiency of gene silencing. The double-stranded region may comprise one or two RNA molecules, transcribed from either one DNA region or two. The presence of the double stranded molecule is thought to trigger a response from an endogenous plant system that destroys both the double stranded RNA and also the homologous RNA transcript from the target plant gene, efficiently reducing or eliminating the activity of the target gene.

The length of the sense and antisense sequences that hybridise should each be at least 19 contiguous nucleotides, preferably at least 30 or 50 nucleotides, and more preferably at least 100, 200, 500 or 1000 nucleotides. The full-length sequence corresponding to the entire gene transcript may be used. The lengths are most preferably 100-2000 nucleotides. The degree of identity of the sense and antisense sequences to the targeted transcript should be at least 85%, preferably at least 90% and more preferably 95-100%. The RNA molecule may of course comprise unrelated sequences which may function to stabilize the molecule. The RNA molecule may be expressed under the control of a RNA polymerase II or RNA polymerase III promoter. Examples of the latter include tRNA or snRNA promoters.

microRNA

MicroRNA regulation is a clearly specialized branch of the RNA silencing pathway that evolved towards gene regulation, diverging from conventional RNAi/PTGS. MicroRNAs are a specific class of small RNAs that are encoded in gene-like elements organized in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RISC-like complexes containing a particular subset of Argonaute proteins that exert sequence-specific gene repression (see, for example, Millar and Waterhouse, 2005; Pasquinelli et al., 2005; Almeida and Allshire, 2005). In an embodiment, the microRNA has 21 consecutive nucleotides of which at least 20 nucleotides, preferably all 21 nucleotides, are identical in sequence to the complement of 21 consecutive nucleotides of the transcribed region of the target gene. That is, the microRNA can tolerate 1 mismatched nucleotide in the sequence of 21 nucleotides, but preferably is identical to the complement of the region of the target gene. The remainder of the stem-looped precursor RNA to the microRNA may be unrelated in sequence to the target gene, and is preferably related in sequence to, or corresponds to, a naturally occurring microRNA precursor.

### Cosuppression

Another molecular biological approach that may be used is co-suppression. The mechanism of co-suppression is not well understood but is thought to involve post-transcriptional gene silencing (PTGS) and in that regard may be very similar to many examples of antisense suppression. It involves introducing an extra copy of a gene or a fragment thereof into a plant in the sense orientation with respect to a promoter for its expression. The size of the sense fragment, its correspondence to target gene regions, and its degree of sequence identity to the target gene are as for the antisense sequences described above. In some instances the additional copy of the gene sequence interferes with the expression of the target plant gene. Reference is made to WO 97/20936 and EP 0465572 for methods of implementing co-suppression approaches.

### Gene Constructs and Vectors

One embodiment of the present invention includes a recombinant (chimeric) vector, which includes at least one isolated polynucleotide molecule encoding a polypeptide/enzyme defined herein, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

One type of recombinant vector comprises a nucleic acid molecule of the present invention operatively linked to an expression vector. As indicated above, the phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, other animal, and plant cells. Preferred expression vectors of the present invention can direct gene expression in yeast, animal or plant cells.

In particular, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art.

Another embodiment of the present invention includes a recombinant cell comprising a host cell transformed with one or more recombinant molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules can remain extrachromosomal or can integrate into one or more sites within a chromosome of the transformed (i.e., recombinant) cell in such a manner that their ability to be expressed is retained.

Trans Genic Plants and Parts thereof

The term “plant” as used herein as a noun refers to whole plants, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plants provided by or contemplated for use in the practice of the present invention include both monocotyledons and dicotyledons. In preferred embodiments, the plants of the present invention are crop plants (for example, cereals and pulses, maize, wheat, potatoes, tapioca, rice, sorghum, millet, cassava, barley, or pea), or other legumes. The plants may be grown for production of edible roots, tubers, leaves, stems, flowers or fruit. The plants may be vegetables or ornamental plants. The plants of the invention may be: corn (Zea mays), canola (Brassica napus, Brassica rapa ssp.), flax (Linum usitatissimum), alfalfa (Medicago sativa), rice (Oryza sativa), rye (Secale cerale), sorghum (Sorghum bicolour, Sorghum vulgare), sunflower (Helianthus annus), wheat (Tritium aestivum), soybean (Glycine max), tobacco (Nicotiana tabacum), potato (Solanum tuberosum), peanuts (Arachis hypogaea), cotton (Gossypium hirsutum), sweet potato (Lopmoea batatus), cassava (Manihot esculenta), coffee (Cofea spp.), coconut (Cocos nucifera), pineapple (Anana comosus), citris tree (Citrus spp.), cocoa (Theobroma cacao), tea (Camellia senensis), banana (Musa spp.), avocado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, or barley.

Grain plants that provide seeds of interest include oil-seed plants and leguminous plants. Seeds of interest include grain seeds, such as corn, wheat, barley, rice, sorghum, rye, etc. Leguminous plants include beans and peas. Beans include guar, locust bean, fenugreek, soybean, garden beans, cowpea, mungbean, lima bean, fava bean, lentils, chickpea, etc.

In one embodiment, the plant is an oilseed plant, preferably an oilseed crop plant. As used herein, an “oilseed plant” is a plant species used for the commercial production of oils from the seeds of the plant. The plant may produce high levels of oil in its fruit, such as olive, oil palm or coconut. Preferably, the oilseed plant is Brassica sp., Gossypium hirsutum, Linum usitatissimum, Helianthus sp., Carthamus tinctorius, Glycine max, Zea mays or Arabidopsis thaliana. More preferably, the oilseed plant is Linum usitatissimum or Carthamus tinctorius.

Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

In a preferred embodiment, the transgenic plants are homozygous for each and every exogenous polynucleotide that has been introduced (transgene) so that their progeny do not segregate for the desired phenotype. The transgenic plants may also be heterozygous for the introduced transgene(s), such as, for example, in F1 progeny which have been grown from hybrid seed. Such plants may provide advantages such as hybrid vigour, well known in the art.

In addition to other transgenes already mentioned, the transgenic plants may also comprise further transgenes involved in the production of LC-PUFAs such as, but not limited to, a Δ6 desaturase, a Δ9 elongase, a Δ8 desaturase, a Δ6 elongase, a Δ5 desaturase with activity on a 20:3 substrate, an omega-desaturase, a Δ9 elongase, a Δ4 desaturase, a Δ7 elongase and/or members of the polyketide synthase pathway. Examples of such enzymes are known in the art and include those described in WO 05/103253 (see, for example, Table 1 of WO 05/103253).

A polynucleotide of the present invention may be expressed constitutively in the transgenic plants during all stages of development. Depending on the use of the plant or plant organs, the polypeptides may be expressed in a stage-specific manner. Furthermore, the polynucleotides may be expressed tissue-specifically.

Regulatory sequences which are known or are found to cause expression of a gene encoding a polypeptide of interest in plants may be used in the present invention. The choice of the regulatory sequences used depends on the target plant and/or target organ of interest. Such regulatory sequences may be obtained from plants or plant viruses, or may be chemically synthesized. Such regulatory sequences are well known to those skilled in the art.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

A number of constitutive promoters that are active in plant cells have been described. Suitable promoters for constitutive expression in plants include, but are not limited to, the cauliflower mosaic virus (CaMV) 35S promoter, the Figwort mosaic virus (FMV) 35S, the sugarcane bacilliform virus promoter, the commelina yellow mottle virus promoter, the light-inducible promoter from the small subunit of the ribulose-1,5-bis-phosphate carboxylase, the rice cytosolic triosephosphate isomerase promoter, the adenine phosphoribosyltransferase promoter of Arabidopsi, the rice actin 1 gene promoter, the mannopine synthase and octopine synthase promoters, the Adh promoter, the sucrose synthase promoter, the R gene complex promoter, and the chlorophyll α/β binding protein gene promoter. These promoters have been used to create DNA vectors that have been expressed in plants; see, e.g., PCT publication WO 8402913. All of these promoters have been used to create various types of plant-expressible recombinant DNA vectors.

For the purpose of expression in tissues of the plant such as seed, particularly seed of an oilseed plant such as of soybean, canola, other Brassicas, cotton, Zea mays, sunflower, safflower, or flax, it is preferred that the promoters utilized in the present invention have relatively high expression in the seed before and/or during production of fatty acids for accumulation and storage in the seed. The promoter for β-conglycinin or other seed-specific promoters such as the linin, napin and phaseolin promoters, can be used.

In a preferred embodiment, the promoter directs expression in tissues and organs in which fatty acid and oil biosynthesis take place, particularly in seed cells such as endosperm cells and cells of the developing embryo. Promoters which are suitable are the oilseed rape napin gene promoter (U.S. Pat. No. 5,608,152), the Vicia faba USP promoter (Baumlein et al., 1991), the Arabidopsis oleosin promoter (WO 98/45461), the Phaseolus vulgaris phaseolin promoter (U.S. Pat. No. 5,504,200), the Brassica Bce4 promoter (WO 91/13980) or the legumin B4 promoter (Baumlein et al., 1992), and promoters which lead to the seed-specific expression in monocots such as maize, barley, wheat, rye, rice and the like. Notable promoters which are suitable are the barley 1pt2 or 1pt1 gene promoter (WO 95/15389 and WO 95/23230) or the promoters described in WO 99/16890 Other promoters include those described by Broun et al. (1998) and US 20030159173.

The 5′ non-translated leader sequence can be derived from the promoter selected to express the heterologous gene sequence of the polynucleotide of the present invention, and can be specifically modified if desired so as to increase translation of mRNA. For a review of optimizing expression of transgenes, see Koziel et al. (1996). The 5′ non-translated regions can also be obtained from plant viral RNAs (Tobacco mosaic virus, Tobacco etch virus, Maize dwarf mosaic virus, Alfalfa mosaic virus, among others) from suitable eukaryotic genes, plant genes (wheat and maize chlorophyll a/b binding protein gene leader), or from a synthetic gene sequence. The present invention is not limited to constructs wherein the non-translated region is derived from the 5′ non-translated sequence that accompanies the promoter sequence. The leader sequence could also be derived from an unrelated promoter or coding sequence. Leader sequences useful in context of the present invention comprise the maize Hsp70 leader (U.S. Pat. No. 5,362,865 and U.S. Pat. No. 5,859,347), and the TMV omega element.

The termination of transcription is accomplished by a 3′ non-translated DNA sequence operably linked in the chimeric vector to the polynucleotide of interest. The 3′ non-translated region of a recombinant DNA molecule contains a polyadenylation signal that functions in plants to cause the addition of adenylate nucleotides to the 3′ end of the RNA. The 3′ non-translated region can be obtained from various genes that are expressed in plant cells. The nopaline synthase 3′ untranslated region, the 3′ untranslated region from pea small subunit Rubisco gene, the 3′ untranslated region from soybean 7S seed storage protein gene are commonly used in this capacity. The 3′ transcribed, non-translated regions containing the polyadenylate signal of Agrobacterium tumor-inducing (Ti) plasmid genes are also suitable.

Four general methods for direct delivery of a gene into cells have been described: (1) chemical methods (Graham et al., 1973); (2) physical methods such as microinjection (Capecchi, 1980); electroporation (see, for example, WO 87/06614, U.S. Pat. No. 5,472,869, 5,384,253, WO 92/09696 and WO 93/21335); and the gene gun (see, for example, U.S. Pat. No. 4,9.45,050 and U.S. Pat. No. 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology, for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. An illustrative embodiment of a method for delivering DNA into Zea mays cells by acceleration is a biolistics a-particle delivery system, that can be used to propel particles coated with DNA through a screen, such as a stainless steel or Nytex screen, onto a filter surface covered with corn cells cultured in suspension. A particle delivery system suitable for use with the present invention is the helium acceleration PDS-1000/He gun available from Bio-Rad Laboratories.

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein one may obtain up to 1000 or more foci of cells transiently expressing a marker gene. The number of cells in a focus that express the exogenous gene product 48 hours post-bombardment often range from one to ten and average one to three.

In bombardment transformation, one may optimize the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of immature embryos.

In another alternative embodiment, plastids can be stably transformed. Methods disclosed for plastid transformation in higher plants include particle gun delivery of DNA containing a selectable marker and targeting of the DNA to the plastid genome through homologous recombination (U.S. Pat. No. 5, 451,513, U.S. Pat. No. 5,545,818, U.S. Pat. No. 5,877,402, U.S. Pat. No. 5,932479, and WO 99/05265).

Accordingly, it is contemplated that one may wish to adjust various aspects of the bombardment parameters in small scale studies to fully optimize the conditions. One may particularly wish to adjust physical parameters such as gap distance, flight distance, tissue distance, and helium pressure. One may also minimize the trauma reduction factors by modifying conditions that influence the physiological state of the recipient cells and that may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. No. 5,177,010, U.S. Pat. No. 5,104,310, U.S. Pat. No. 5,004,863, U.S. Pat. No. 5,159,135). Further, the integration of the T-DNA is a relatively precise process resulting in few rearrangements. The region of DNA to be transferred is defined by the border sequences, and intervening DNA is usually inserted into the plant genome.

Modern Agrobacterium transformation vectors are capable of replication in E. coli as well as Agrobacterium, allowing for convenient manipulations as described (Klee et al., In: Plant DNA Infectious Agents, Hohn and Schell, eds., Springer-Verlag, New York, pp. 179-203 (1985). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A transgenic plant formed using Agrobacterium transformation methods typically contains a single genetic locus on one chromosome. Such transgenic plants can be referred to as being hemizygous for the added gene. More preferred is a transgenic plant that is homozygous for the added gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants for the gene of interest.

It is also to be understood that two different transgenic plants can also be mated to produce offspring that contain two independently segregating exogenous genes. Selfing of appropriate progeny can produce plants that are homozygous for both exogenous genes. Back-crossing to a parental plant and out-crossing with a non-transgenic plant are also contemplated, as is vegetative propagation. Descriptions of other breeding methods that are commonly used for different traits and crops can be found in Fehr, In: Breeding Methods for Cultivar Development, Wilcox J. ed., American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art

(Weissbach et al., In: Methods for Plant Molecular Biology, Academic Press, San Diego, Calif., (1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired exogenous nucleic acid is cultivated using methods well known to one skilled in the art.

Methods for transforming dicots, primarily by use of Agrobacterium tumefaciens, and obtaining transgenic plants have been published for cotton (U.S. Pat. No. 5,004,863, U.S. Pat. No. 5,159,135, U.S. Pat. No. 5,518,908); soybean (U.S. Pat. No. 5,569,834, U.S. Pat. No. 5,416,011); Brassica (U.S. Pat. No. 5,463,174); peanut (Cheng et al., 1996); and pea (Grant et al., 1995).

Methods for transformation of cereal plants such as wheat and barley for introducing genetic variation into the plant by introduction of an exogenous nucleic acid and for regeneration of plants from protoplasts or immature plant embryos are well known in the art, see for example, Canadian Patent Application No. 2,092,588, Australian Patent Application No 61781/94, Australian Patent No 667939, U.S. Pat. No. 6,100,447, International Patent Application PCT/US97/10621, U.S. Pat. No. 5,589,617, U.S. Pat. No. 6,541,257, and other methods are set out in Patent specification WO99/14314. Preferably, transgenic wheat or barley plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts.

The regenerable wheat cells are preferably from the scutellum of immature embryos, mature embryos, callus derived from these, or the meristematic tissue.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (Ed. M. J. McPherson and S. G Moller (2000) BIOS Scientific Publishers Ltd, Oxford). PCR can be performed on cDNA obtained from reverse transcribing mRNA isolated from plant cells. However, it will generally be easier if PCR is performed on genomic DNA isolated from a plant.

A primer is an oligonucleotide sequence that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al. (supra) and Sambrook et al. (supra). Sequencing can be carried out by any suitable method, for example, dideoxy sequencing, chemical sequencing or variations thereof. Direct sequencing has the advantage of determining variation in any base pair of a particular sequence.

### Production of Oils

Techniques that are routinely practiced in the art can be used to extract, process, and analyze the oils produced by cells, plants, seeds, etc of the instant invention. Typically, plant seeds are cooked, pressed, and extracted to produce crude oil, which is then degummed, refined, bleached, and deodorized. Generally, techniques for crushing seed are known in the art. For example, oilseeds can be tempered by spraying them with water to raise the moisture content to, e.g., 8.5%, and flaked using a smooth roller with a gap setting of 0.23 to 0.27 mm. Depending on the type of seed, water may not be added prior to crushing. Application of heat deactivates enzymes, facilitates further cell rupturing, coalesces the oil droplets, and agglomerates protein particles, all of which facilitate the extraction process.

The majority of the seed oil is released by passage through a screw press. Cakes expelled from the screw press are then solvent extracted, e.g., with hexane, using a heat traced column. Alternatively, crude oil produced by the pressing operation can be passed through a settling tank with a slotted wire drainage top to remove the solids that are expressed with the oil during the pressing operation. The clarified oil can be passed through a plate and frame filter to remove any remaining fine solid particles. If desired, the oil recovered from the extraction process can be combined with the clarified oil to produce a blended crude oil.

Once the solvent is stripped from the crude oil, the pressed and extracted portions are combined and subjected to normal oil processing procedures (i.e., degumming, caustic refining, bleaching, and deodorization). Degumming can be performed by addition of concentrated phosphoric acid to the crude oil to convert non-hydratable phosphatides to a hydratable form, and to chelate minor metals that are present. Gum is separated from the oil by centrifugation. The oil can be refined by addition of a sufficient amount of a sodium hydroxide solution to titrate all of the fatty acids and removing the soaps thus formed.

Deodorization can be performed by heating the oil to 260° C. under vacuum, and slowly introducing steam into the oil at a rate of about 0.1 ml/minute/100 ml of oil. After about 30 minutes of sparging, the oil is allowed to cool under vacuum. The oil is typically transferred to a glass container and flushed with argon before being stored under refrigeration. If the amount of oil is limited, the oil can be placed under vacuum, e.g., in a Parr reactor and heated to 260° C. for the same length of time that it would have been deodorized. This treatment improves the color of the oil and removes a majority of the volatile substances.

### Antibodies

The invention also provides antibodies, such as monoclonal or polyclonal antibodies, to polypeptides of the invention or fragments thereof. Thus, the present invention further provides a process for the production of monoclonal or polyclonal antibodies to polypeptides of the invention.

The term “binds specifically” refers to the ability of the antibody to bind to at least one protein of the present invention but not other proteins present in a recombinant (transgenic) cell, particularly a recombinant plant or yeast cell of the invention.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv).

### EXAMPLES

### Example 1

### Materials and Methods

Construction of Lemon Thyme cDNA Library

Total RNA was isolated from developing seeds using Trizol reagent according to the instructions of the supplier. Messenger RNA was purified from total RNA using an Oligotex mRNA kit (Qiagen). First strand cDNA was synthesised from 5 ng mRNA using an oligo-dT primer supplied with the ZAP-cDNA synthesis kit (Stratagene≦Catalogue No. 200400) and reverse transcriptase SuperscriptIII (Invitrogen). Double stranded cDNA was ligated to EcoRI/XhoI adaptors and from this a library was constructed using the ZAP-cDNA synthesis kit according to the suppliers' instructions.

Yeast Culturing and Feeding with Precursor Fatty Acids

Plasmids were introduced into yeast by a standard heat shock method and transformants selected on yeast synthetic drop out (SD) medium plates containing 2% glucose. Experimental cultures were inoculated the yeast cells carrying different plasmids in SD medium containing 2% glucose, 1% NP-40, to an initial 0D₆₀₀ of about 0.3. Cultures were grown at 30° C. with shaking until OD₆₀₀ was approximately 1.0. The cells were harvested by centrifugation and washed with sterile water, then resuspended into the same volume of synthetic media (SD) with 2% galactose (SG) instead of glucose. Selected precursor fatty acids were added to a final concentration of 0.5 mM at the presence of 1% NP-40. Cultures were incubated at 30° C. with shaking for a further 48 hours prior to harvesting by centrifugation. Cell pellets were washed with 1% NP-40, 0.5% NP-40 and water to remove any unincorporated fatty acids from the surface of the cells.

### Plant Transformation

Arabidopsis thaliana Columbia and fad2/fael double mutant were used in transformation experiments. All Arabidopsis transformations were done by spraying flower buds with suspensions of A. tumefaciens (AGL1 strain) carrying the various expression constructs made as described. Seeds were collected from the treated plants (T₀ generation) at maturity. Primary transformants (T₁ generation) were identified by plating the seeds on medium containing kanamycin, where expression of antibiotic resistance was indicative of presence of the Kan selectable marker gene and therefore of transformation (Stoutjesdijk et al., 2002). All transgenic Arabidopsis plants were grown in a greenhouse under natural day-length at controlled temperatures of 24° C. in the daylight hours and 18° C. during the night. Selfed seeds (T₂ generation) from the T₁ plants were harvested and the seed fatty acid composition was analysed by gas-liquid chromatography (GC) by standard methods. For segregation studies, individual T₂ seeds were planted, the T₂ plants grown to maturity, and T₃ seeds were harvested and analysed for antibiotic resistance and fatty acid composition of seed oil by GC.

Fatty Acid methyl esters (FAME) Preparation

Fatty acid methyl esters (FAME) were formed by transesterification of the total fatty acids in yeast cells (pellets after cenrtrifugation) or Arabidopsis seeds by adding 300 μL of 1% NaMeOH in methanol at room temperature for 20 min, then added 300 μL of 1M NaCl. FAMEs were extracted with 300 uL of hexane and analysed by GC and GC-MS. Characteristic analysis of hydroxy FAME was also done by converting aliquots of dried FAME by 20 ul of 1% TCMS in BSTFA (Supelco, Catalogue #33154-U) into TMS derivatives at 80° C. for 30 min, then run on gas chromatography-mass spectrometry.

### Capillary Gas-Liquid Chromatography (GC)

FAME were analysed with an Agilent 6890 gas chromatograph fitted with 6980 series automatic injectors respectively and a flame-ionization detector (FID). Injector and detector temperatures used were 270° C. and 280° C. respectively. FAME samples were injected at 170° C. onto a BPX70 polar capillary column (SGE; 60 m×0.25 mm i.d.; 0.25 μm film thickness). After 2 min, the oven temperature was raised to 220° C. at 25min⁻¹, then to 224° C. at 1° C. min⁻¹, then to a final temperature of 240° C. at 2° C. min⁻¹ where it was kept for 4 min. Helium was the carrier gas with a column head pressure of 45 psi and the purge opened 2 min after injection. Identification of peaks was based on comparison of relative retention time data with standard FAMEs. For quantification, Chemstation (Agilent) was used to integrate peak areas.

### Gas Chromatography-Mass Spectrometry (GC-MS)

GC-MS was carried out on a Finnigan Polaris Q and Trace GC2000 GC-MS ion-trap fitted with on-column injection. Samples were injected using an AS3000 auto sampler onto a retention gap attached to a BPX70 polar capillary column (SGE; 30 m×0.25 mm i.d.; 0.25 μm film thickness). The initial temperature of 60° C. was held for 1 min, followed by temperature programming at 30° C.min⁻¹ to 120° C. then at 9° C.min⁻¹ to 250° C. where it was held for 1 min. Helium was used as the carrier gas. Mass spectra were acquired and processed with Xcalibur™ software.

### Purification of Lipids by Soxhlet Extraction

The material obtained through fermentation was placed inside a thimble made from thick filter paper, which was loaded into the main chamber of a Soxhlet extractor. The Soxhlet extractor was placed onto a flask containing the extraction solvent (hexane or chloroform). The Soxhlet was then equipped with a condenser. The solvent was heated to reflux. The solvent vapour flooded into the chamber housing the thimble of fermentation material. The condenser ensures that any solvent vapour cools, and drips back down into the chamber housing the solid material. The chamber containing the solid material slowly filled with solvent. When the Soxhlet chamber is almost full, the chamber is automatically emptied by a siphon side arm, with the solvent running back down to the distillation flask. This cycle was allowed to repeat over 48 hours. After extraction the solvent was removed under reduced pressure, yielding the extracted compound. The non-soluble portion of the extracted solid remained in the thimble, and was usually discarded.

### NMR Methods

Proton NMR spectra of purified fatty acids were obtained on Bruker AV400 and Bruker AV200 spectrometer, operating at 400 MHz and 200 MHz. All spectra were obtained at 23° C. unless specified. Chemical shifts are reported in parts per million (ppm) on the δ scale and relative to the chloroform peak at 7.26 ppm (¹H) or the TMS peak at 0.00 ppm (¹H). All starting solvents and reagents were obtained commercially unless otherwise stated. Removal of solvents “under reduced pressure” refers to the process of bulk solvent removal by rotary evaporation (low vacuum pump) followed by application of high vacuum pump (oil pump) for a minimum of 30 min. Chloroform and Hexane were obtained from a solvent dispensing system under an inert atmosphere.

### Example 2

### Isolation of Arabidopsis thaliana FAH Genes and Expression in Yeast Cells

In an attempt to identify plant or fungal genes which might be candidates for Δ2-hydroxylases active on fatty acids, particularly for phospholipid linked fatty acids, the inventors considered known fatty acid hydroxylase genes from other organisms. The yeast protein FAH1p encoded by the Scs7 gene catalyses the Δ2-hydroxylation (also known as 2-hydroxylation or a-hydroxylation) of sphingolipid-associated very long chain fatty acids. These very long chain fatty acids mostly have acyl chains of 26 carbons (C26). An Arabidopsis homologue of FAH1p designated AtFAH1 (also denoted as ArathFAH1 herein), encoded by the At2g34770 gene, has been shown to complement an scs7 mutation in yeast (Mitchell and Martin, 1997) and presumably catalyses the same reaction.

When the Arabidopsis genome was queried for genes encoding proteins with homology to AtFAH1, surprisingly a second gene (At4g20870) was noticed, encoding a protein designated herein as AtFAH2. This gene was expressed predominantly in pollen and was thought to be involved in the hydroxylation of acyl chains in sphingolipid.

Cloning of Arabidopsis thaliana AtFAH1 and AtFAH2

AtFAH1 and AtFAH2 genes were amplified from Arabidopsis thaliana Columbia flower cDNA with proof-reading polymerase Pfu polymerase (Stratagene) and primers:

(SEQ ID NO: 25)
A2-34770-OF 5′-TTCCGTCTCGAGTCGCTGA-3′
and
(SEQ ID NO: 26)
A2-34770-OR 5′-CTTTCTAGACTATTGCTCTTTTCTGGGGG-3′
for FAH1
(SEQ ID NO: 27)
A4-20870-OF 5′-TTTGGATCCTCTAAGAAACAATCAGTCTC-3′
and
(SEQ ID NO: 28)
A4-20870-OR 5′-GCATCTAGAAATCCGATTTAGCTCTTCT-3′
for FAH2

and inserted into a yeast expression vector pYES2 (Invitrogen) after restriction digestion of PCR products, resulting in plasmids pYES2-AtFAH1 and pYES2-AtFAH2. After confirming the nucleotide sequence of the coding region, the plasmids were transformed into yeast cells. The genes were also cleaved out by EcoRV-XbaI or BamHI-XbaI, and subcloned into binary vector pGNAP (Lee et al., 1998), generating plasmid pGNAP-AtFAH1 and pGNAP-AtFAH2, for expression in transgenic plants by the methods described in Example 1.
Expression of Arabidopsis thaliana AtFAH1 and AtFAH2 in Yeast

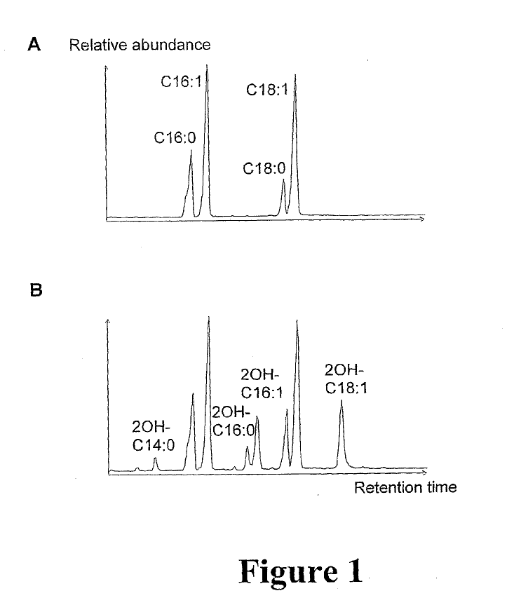

Yeast expression plasmids pYES2-AtFAH1 and pYES2-AtFAH2A were transformed into yeast wild type strains S288C and YPH499 or olel mutant strain. The later results showed that YPH499 was the best strain of those tested for this assay, therefore it was used in the following experiments. Wild type yeast cells have four major fatty acids, C16:0, C16:0^{Δ9}, C18:0, C18:1^{Δ9}, with low levels of C14:0, C26:0 as minor components. From the GC analysis, the fatty acid composition of the yeast YPH499 cells expressing AtFAH1 gene was the same as yeast cells expressing pYES2 vector without an additional gene insert. In contrast, the cells expressing pYES2 containing AtFAH2 gene produced novel fatty acids as shown by peaks for 2OH-C14:0, 2OH-C16:0, 2OH-C16:1^{Δ9} and 2OH-C18:1^{Δ9} (FIG. 1 and Table 2). The identities of these novel fatty acids were confirmed by GC-MS using TMS derivatives. The efficiency of conversion of oleic to 2-hydroxyoleic acid was greater than 10%.

Larger Scale Biotransformation of Oleic Acid to 2-hydroxyoleic Acid using Whole Cells

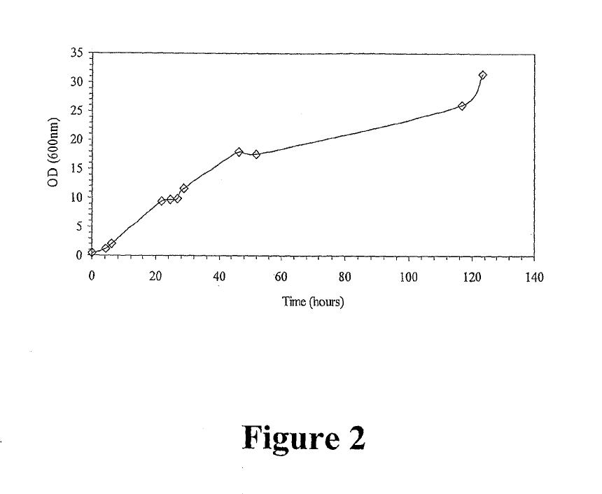

In order to convert oleic acid into 2-hydroxyoleic acid in a larger-scale fermentation system, cultures of yeast (Saccharomyces cerevisiae) strain YPH499 containing vector pYES₂::208870 (FAH2) and therefore expressing the AtFAH2 fatty acid hydroxylase 2 gene were grown in defined medium in a 2 litre stirred tank reactor. The strain was grown at 30° C. and the culture was maintained at pH 5.0 by the automatic addition of either 10% phosphoric acid or 10% ammonia solution. The culture was sparged with air at a flow rate of 0.45 L min⁻¹. The defined medium was selective dropout (SD) medium that lacked uracil, and was supplemented with lysine, adenine, tryptophan, histidine and leucine. The initial carbon source was 20 g L⁻¹ glucose. FAH2 protein expression was induced 24 hours after inoculation by addition of galactose to a final concentration of 20 g L⁻¹. A volume (9 mL) of 20% (v/v) of oleic acid dissolved in ethanol was added to the culture 2.4 hours after the addition of galactose. Cell growth is shown in FIG. 2.

TABLE 2
Fatty acid composition (%) of yeast cells expressing pYES2-
AtFAH2 and the conversion rates of substrates to products

| | | 2OH— | | | 2OH— | 2OH— | | | 2OH— |
|---|---|---|---|---|---|---|---|---|---|
| FA | C14:0 | C14:0 | C16:0 | C16:1 | C16:0 | C16:1 | C18:0 | C18:1 | C18:1 |
| Vector | 0.4 | 0.0 | 19.8 | 39.9 | 0.0 | 0.0 | 7.0 | 32.9 | 0.0 |
| AtFAH2 | 1.0 | 0.8 | 19.3 | 31.3 | 1.5 | 3.8 | 8.8 | 28.8 | 4.8 |
| Conversion | | 43.4% | | | 7.1% | 10.7% | | | 14.3% |

The culture was grown for a further 138 hours after the addition of galactose and then harvested by centrifugation. The cell pellet was resuspended in water, acidified with hydrochloric acid (pH 1) and lipids extracted 3 times with chloroform. The combined organic layer was evaporated under reduced pressure and the successful conversion of oleic acid to 2-hydroxyoleic acid was confirmed using standard analytical techniques. Electrospray mass spectrometry was used to confirm the presence of both oleic and 2-hydroxyoleic acids in the chloroform extract. The fatty acids in the crude product mixture were converted into the methyl esters using methanol and TMS-chloride. The methyl ester mixture (430 mg) was purified through column chromatography (SiO₂/ethyl acetate/hexane) and showed the presence of 81.4% oleic acid methyl ester, 9.3% 2-hydroxyoleic acid methyl ester, 3.3% column-bound impurities and 3.7% unbound impurities. This therefore represented an efficiency for conversion of oleic acid to 2-hydroxyoleic acid of at least 10%.

### Example 3

### Identification and Characterisation of FAH2 Gene Family

On the basis of the data in Example 2, the inventors considered whether other plant or fungal enzymes catalyzing the Δ2-hydroxylation of C18 fatty acids might be homologous to the archetypal yeast and Arabidopsis proteins. The plant EST database was searched with the archetypal yeast and Arabidopsis FAH1 sequences using both amino acid and nucleotide sequences, and then searched iteratively using the homologous sequences. More than fifty plants sequences were identified which were, or encoded proteins which were, homologous to the yeast FAH1p and Arabidopsis FAH1. These were represented by partial length EST sequences which did not encode full length proteins. However, contigs covering entire coding sequences (some examples shown as SEQ ID NOs: 14 to 24) were assembled for individual plants species by identifying overlapping ESTs, and the contigs translated into amino acid sequences (some examples shown as SEQ ID NOs: 2 to 12).

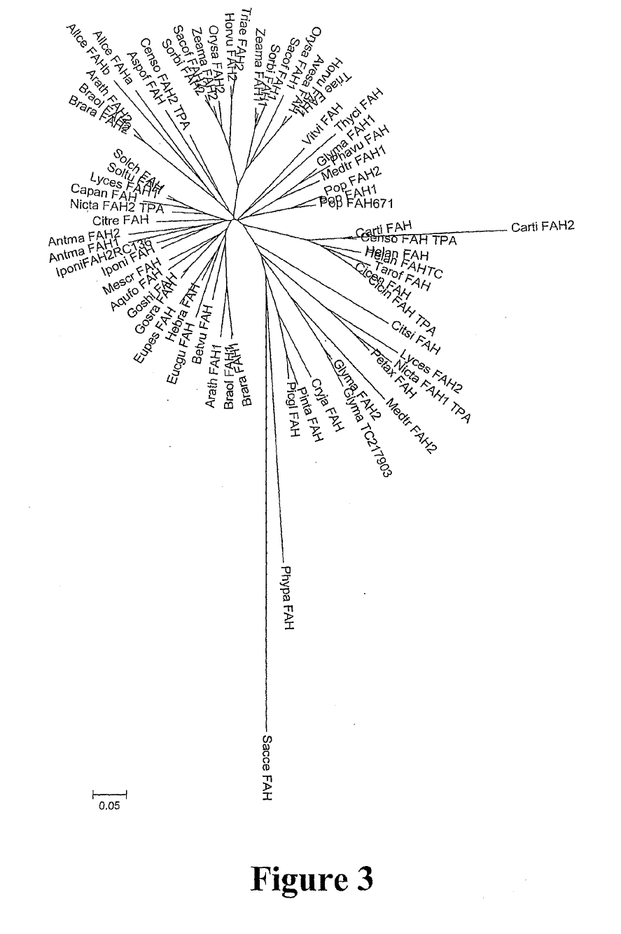

The amino acid sequences were aligned, together with a representative framework of the more distantly related fatty acid desaturase sequences encoded by the Arabidopsis genome as well as the archetypal yeast FAH1p sequence, and a phylogenetic tree constructed from the alignment (FIG. 3). The proteins that clustered together with the yeast FAH1p sequence and separately from the other Arabidopsis desaturase sequences were considered to be candidate Δ2-hydroxylases and were used for a new amino acid sequence alignment. Although all fungal Δ2-hydroxylases have an N-terminal Cyt B5 domain, none of the plant Δ2-hydroxylase-like sequences identified herein contained a cytB5 domain. The two Arabidopsis proteins, AtFAH1 and AtFAH2 (At4g20870) were present in the 2-hydroxylase cluster and clearly belonged to this family.

### Example 4

### Isolation of Gene Encoding FAH from Lemon Thyme (Thymus citriodorus)

When all of the Δ2-hydroxylase-like proteins which were distinct from other plant desaturase-like sequences were aligned, it was observed that there were nine conserved motifs within the proteins. When the Arabidopsis genome sequence was interogated with these amino acid sequences only two proteins were found, AtFAH1 and AtFAH2 mentioned above, showing that the motifs identified were specific for the fatty acid hydroxylase cluster identified herein (Table 3).

TABLE 3
Conserved motifs from FAH proteins.

| | | Peptide position |
|---|---|---|
| Motif | Peptide sequence in AtFAH1 | AtFAH1 |
| Motif 1 | VDL[D/N]KPLVFQVGHL | 8-21 |
| | (SEQ ID NO: 40) | |
| Motif 2 | WVHQPIVSKEGPRFF | 28-42 |
| | (SEQ ID NO: 41) | |
| Motif 3 | WWAIPTIWLPV | 55-65 |
| | (SEQ ID NO: 42) | |
| Motif 4 | WTLLEYTLHRFLFHI KTKSYW | 94-114 |
| | (SEQ ID NO: 43) | |
| Motif 5 | HYLLHGCHHKHP | 119-130 |
| | (SEQ ID NO: 44) | |
| Motif 6 | RLVFPPAAA[T/A]AIL | 135-147 |
| | (SEQ ID NO: 45) | |
| Motif 7 | GYVMYDCTHYYLHHGQP | 173-189 |
| | (SEQ ID NO: 46) | |
| Motif 8 | KKYHLNHHFRIQ | 198-209 |
| | (SEQ ID NO: 47) | |
| Motif 9 | KGFGITSSLWD | 211-221 |
| | (SEQ ID NO: 48) | |

In order to clone an FAH gene from lemon thyme, degenerate oligonucleotides based on these motifs and biased towards thyme-related (Lamiid) plants (Solanum tuberosum, Lycopersicon esculentum and Ipomoea nil) were designed and made (Table 4). These were used in pairwise combinations in attempts to amplify gene fragments. Some of these resulted in amplified products. These were used as probes to isolate a cDNA sequence from a cDNA library from Thymus citriodorus. One full length clone was isolated. Its nucleotide sequence is shown as SEQ ID NO:13 and the predicted amino acid sequence is shown as SEQ ID NO: 1. This gene was designated TcFAH (also denoted herein as ThyciFAH).

TABLE 4
Degenerate oligonucleotide sequences coding
for conserved motifs in FAH gene family

FAH_Forl
GTIGAYCTIRAYAARCCICTIGTITTYCARGTIGGICAYCT
(SEQ ID NO: 29)
FAH_For2
TICAYCARCCIATHGTITCIAARGARGGICCICGITTYTTY
(SEQ ID NO: 30)
FAH_For3
GTGGGCIATHCCIACIATHTGGCTICCIGT (SEQ ID NO: 31)
FAH_For4
TGGACICTIATGGARTAYACICTICAYCGITTYCTITTYCAYATHAA
(SEQ ID NO: 32)
FAH_Rev4
CCAATAIGAITTIGTITTTATATGAAAIAGAAAICGATGIAGIGTATAT
TCCAT (SEQ ID NO: 33)
FAH_For5
CAYTAYCTICTICAYGGITGYCAYCAYAARCAYCC
(SEQ ID NO: 34)
FAH_Rev5
GGRTGYTTRTGRTGRCAICCRTGIAGIAGRTARTG
(SEQ ID NO: 35)
FAH_Rev6
AGDATIGCIGCIGCIGCIGCIGGIGGRAA (SEQ ID NO: 36)
FAH_Rev7
GGYTGICCRTGRTGIAGRTARTARTGIGTIACRTCRTACATIAC
(SEQ ID NO: 37)
FAH_Rev8
YTGDATICGRAARTGRTGRTTIAGRTGRTAYTTYTT
(SEQ ID NO: 38)
FAH_Rev9
RTCCCARAAIGAIGAIGTDATICCRAAICCYTT (SEQ ID NO: 39)

The protein coding region of TcFAH was inserted into yeast and plant expression vectors in the same manner as for the Arabidopsis genes described above.

Expression of thyme LtFAH2 in Yeast

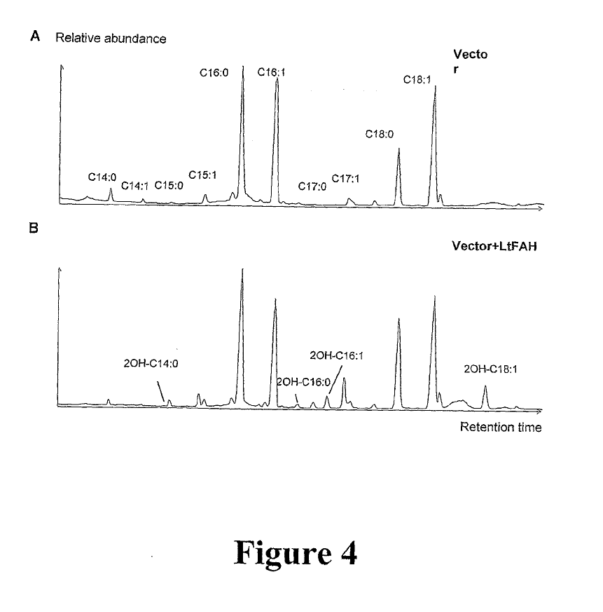

When the protein coding region of LtFAH2 was expressed in yeast cells and the resultant fatty acids analysed, it was observed that the cells produced 2OH-C14:0, 2OH-C16:0, 2OH-C16:1^{Δ9} and 2OH-C18:1^{Δ9} (FIG. 4 and Table 5) in a similar fashion to AtFAH2.

Several vector/host systems for yeast expression were tested for higher hydroxylating activity of oleic acid as a substrate. The product fatty acids were extracted from the fermentation products and analysed by NMR as described in Example 1. The LtFAH2 gene inserted in the vector pYEX.Bx in yeast strain IMY22 yielded greater than 50% hydroxylation of oleic acid to 2OH-oleic acid, which was substantially increased compared to the same gene in pYES2 (galactose-inducible expression) in yeast strainYPH499. pYEX.Bx was described in Australian Patent Application No. 88/15845, and contains an expression cassette under the control of the copper-inducible promoter from the CUP1 gene, and provided a low cost fermentation system.

The LtFAH2 gene was more effective in the fermentation system than the Arabidopsis FAH2 gene which yielded a conversion efficiency of oleic acid of about 30%.

### Example 5

### Expression of AtFAH2 and LtFAH2 in Transgenic Plants

Seeds of an untransformed Arabidopsis thaliana fad2/fael mutant plant had trace amount (<0.1%) of 2OH-C18:1 (Table 3). This may have been due to expression of the endogenous AtFAH2 gene at very low levels in tissues such as seeds in addition to the major site of expression in pollen. When transgenic lines specifically expressing AtFAH2 in fad2/fael seeds were examined, at least one line was shown (Table 6) to contain an increased level of 2OH-C 18:1 in seed fatty acid.

It is expected that when other genes in the gene family are expressed in yeast or plant cells, they will also catalyse production of 2-hydroxyl fatty acids such as 2-hydroxy oleic acid and 2-hydroxylinoleic acid in the developing seed, with resultant accumulation in the seed oil.

TABLE 5
Fatty acid composition (%) of yeast cells expressing pYES-LtFAH2 and the conversion rates of substrates to products

| | | | 2OH— | | | | | 2OH— | | 2OH— | | | | 2OH— |
|---|---|---|---|---|---|---|---|---|---|---|---|---|---|---|
| FA | C14:0 | C14:1 | C14:0 | C15:0 | C15:1 | C16:0 | C16:1 | C16:0 | C17:0 | C16:1 | C17:1 | C18:0 | C18:1 | C18:1 |
| Vector | 3.7 | 1.3 | 0 | 0.3 | 0 | 24.2 | 31.9 | 0 | 0.1 | 0 | 0 | 10 | 28.5 | 0 |
| LtFAH2 | 1.3 | 0.3 | 0.02 | 1 | 3 | 24.4 | 22.8 | 0.3 | 0.9 | 0.9 | 6.6 | 13.6 | 23.3 | 1.6 |
| Conversion | | | 1.6% | | | | | 1.3% | | 3.7% | | | | 6.40% |

TABLE 6
Seed fatty acid composition (%) of fad2/fae1 mutant expressing AtFAH2

| | | | 2OH— | | | 2OH— | | | |
|---|---|---|---|---|---|---|---|---|---|
| FA | C16:0 | C16:1 | C16:0 | C18:0 | C18:1 | C18:1 | C18:2 | C18:3 | C20:0 |
| fad2/fae1 | 17.4 | 2.0 | 0.1 | 6.1 | 59.7 | 0.1 | 2.8 | 9.5 | 2.3 |
| fad2/fae1 (AtFAH2) | 16.9 | 2.2 | 0.1 | 7.8 | 54.8 | 1.3 | 2.6 | 10.3 | 3.9 |

### Example 6

### Hydroxylase Activity on Various Fatty Acid Substrates

Biotransformation of hexadeca-7,9-diynoic acid to 2-hydroxy-hexadeca-7,9-diynoic Acid and hexadeca-10,12-diynoic Acid to 2-hydroxy-hexadeca-7,9-diynoic Acid using Cell Lysate

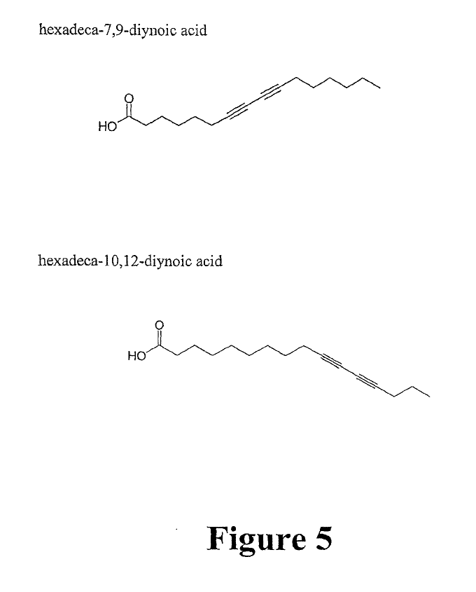

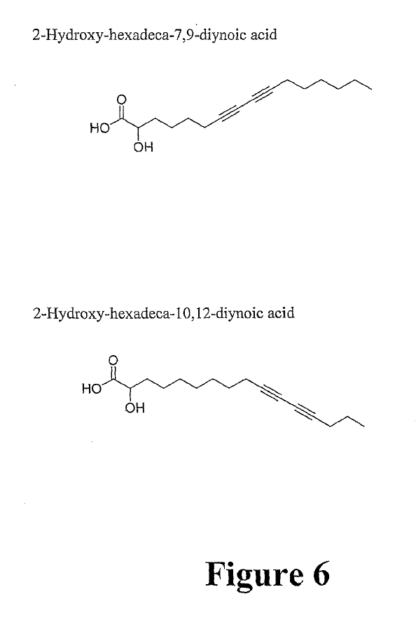

Yeast cell lysates were used to produce 2-hydroxy derivatives of the diyne fatty acids shown in FIGS. 5 and 6.

Yeast strain S. cerevisiae YPH499 containing pYES₂::208870 (FAH2) was grown in batch cultures as described above and protein expression induced 27 hours after inoculation by addition of galactose to a final concentration of 20 g L⁻¹. The cells were harvested by centrifugation 43 hours after the addition of galactose. The yeast cell pellet (25 g wet weight) was resuspended in 50 mL of 0.1 M Tris-HCl (pH 7.5) containing 1 mM PMSF and the cells lysed by two passes through an homogeniser operating at 1000 bar. The fatty acids hexadeca-7,9-diynoic acid and hexadeca-10,12-diynoic acid were dissolved in 2M Tris-HCl, pH 10.3 at a concentration of 20 mg mL⁻¹. Aliquots (5 mL) of the fatty acid solutions were added to 30 mL aliquots of homogenate. The mixture was adjusted to pH 6.0 and incubation at 22° C. for 40 hours with slow agitation. At the completion of the incubation the cell lysate/fatty acid mixtures were extracted with chloroform for analysis (as described above). The successful conversion of both diyne fatty acids was also confirmed using a standard LCMS technique.

Trace amounts of 2-hydroxy-hexadeca-7,9-diynoic acid were detected and 2% of the extracted material was 2-hydroxy-hexadeca-10,12-diynoic acid.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from US 61/125,437 filed 25 April 2008, the entire contents of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abdullah et al. (1986) Biotechnology 4:1087.

Almeida and Allshire (2005) TRENDS Cell Biol., 15:251-258.

Baumlein et al. (1991) Mol. Gen. Genet. 225:459-467.

Baumlein et al. (1992) Plant J. 2:233-239.

Bourque (1995) Plant Sci. 105:125-149.

Broun and Somerville (1997) Plant Physiol. 113:933-942.

Broun et al. (1998) Plant J. 13:201-210.

Capecchi (1980) Cell 22:479-488.

Cheng et al. (1996) Plant Cell Rep. 15:653-657.

Clapp (1993) Clin. Perinatol. 20:155-168.

Curiel et al. (1992) Hum. Gen. Ther. 3:147-154.

Eglitis et al. (1988) Biotechniques 6:608-614.

Fujimura et al. (1985) Plant Tissue Culture Letters 2:74.

Graham et al. (1973) Virology 54:536-539.

Grant et al. (1995) Plant Cell Rep. 15:254-258.

Harayama (1998). Trends Biotechnol. 16: 76-82.

Haseloff and Gerlach (1988) Nature 334:585-591.

Koziel et al. (1996) Plant. Mol. Biol. 32:393-405.

Langridge et al. (2001) Aust. J. Agric. Res. 52: 1043-1077.

Lee et al. (1998) Science 280:915-918.

Lemieux (2000) Current Genomics 1: 301-311.

Lu et al. (1993) J. Exp. Med. 178:2089-2096.

Millar and Waterhouse (2005) Funct. Integr. Genomics 5:129-135.

Mitchell and Martin (1997) J. Biol. Chem. 272:28281.

Needleman and Wunsch (1970) J. Mol. Biol. 48:443-453.

Pasquinelli et al. (2005) Curr. Opin. Genet. Develop., 15:200-205.

Perriman et al. (1992) Gene 113:157-163.

Saha et al. (2006) Plant Physiol. 141:1533-1543.

Senior (1998) Biotech. Genet. Engin. Revs. 15: 79-119.

Shippy et al. (1999) Mol. Biotech. 12:117-129.

Smith et al. (2000) Nature 407:319-320.

Smith et al, (2003) Planta 217:507-516.

Stoutjesdijk et al. (2002) Plant Physiol. 129:1723-1731.

Toriyama et al. (1986) Theor. Appl. Genet. 205:34.

van de Loo et al. (1995) Proc Natl Acad Sci U S A. 92:6743-7.

Wagner et al. (1992) Proc. Natl. Acad. Sci. USA 89:6099-6103.

Waterhouse et al. (1998) Proc. Natl. Acad. Sci. USA 95:13959-13964.

1. A eukaryotic cell comprising an exogenous nucleic acid encoding a polypeptide with fatty acid Δ2 hydroxylase activity.

2. The eukaryotic cell of claim 1, wherein
i) the hydroxylase has an efficiency of conversion of oleic acid to 2-hydroxyoleic acid of at least 1%, or at least 10%,
ii) the hydroxylase, when expressed in a yeast cell, is capable of converting at least 1% of oleic acid in the yeast cell to 2-hydroxyoleic acid,
iii) the hydroxylase has an efficiency of conversion of palmitic acid to 2-hydroxypalmitic acid of at least 1%, or at least 5%,
iv) the hydroxylase, when expressed in a yeast cell, is capable of converting at least 1% of palmitic acid in the yeast cell to 2-hydroxypalmitic acid,
v) the hydroxylase has an efficiency of conversion of palmitoleic acid to 2-hydroxy palmitoleic acid of at least 1%, or at least 10%,
vi) the hydroxylase, when expressed in a yeast cell, is capable of converting at least 1% of palmitoleic acid in the yeast cell to 2-hydroxyl palmitoleic acid,
vii) the polypeptide is capable of converting oleic acid to 2-hydroxyoleic acid, myristic acid to 2-hydroxymyristic acid, palmitic acid to 2-hydroxypalmitic acid, and/or palmitoleic acid to hydroxy palmitoleic acid,
viii) the polypeptide comprises amino acids having a sequence as set forth in any one of SEQ ID NOs :1, 2 and 4 to 12, a biologically active fragment thereof, or an amino acid sequence which is at least 30% identical to any one or more of the sequences set forth in SEQ ID NOs: 1, 2 and 4 to 12,
ix) the cell comprises an increased level of 2-hydroxy fatty acids relative to a corresponding eukaryotic cell lacking the exogenous nucleic acid,
x) the cell comprises an increased level of 2OH-C14:0, 2OH-C16:0, 2OH-C16:1^{Δ9} and/or 2OH-C18:1^{Δ9} fatty acids relative to a corresponding eukaryotic cell lacking the exogenous nucleic acid, and/or
xi) the cell is a plant cell or a cell suitable for fermentation.

3.-12. (canceled)

13. A method of producing a transgenic cell which is capable of hydroxylating fatty acids at the Δ2 position, the method comprising
i) introducing into a eukaryotic cell an exogenous polynucleotide encoding a fatty acid hydroxylase, and
ii) analysing the cell or a tissue, organ or organism comprising the cell, or progeny thereof, for ability to produce Δ2 hydroxylated fatty acids.

14.-16. (canceled)

17. A process for determining whether a nucleic acid molecule encodes a fatty acid Δ2 hydroxylase, and/or for identifying a nucleic acid molecule encoding a fatty acid Δ2 hydroxylase, comprising:
i) obtaining a nucleic acid molecule operably linked to a promoter, the nucleic acid molecule encoding a polypeptide comprising amino acids having a sequence as set forth in any one of SEQ ID NOs:1, 2 and 4 to 12 or having at least 30% amino acid identity to at least one of SEQ ID NOs 1, 2 and 4 to 12, and/or having a sequence more closely related to SEQ ID NO:2 than to SEQ ID NO:3,
ii) introducing the nucleic acid molecule into a cell or cell-free expression system in which the promoter is active,
iii) determining whether the level of fatty acid Δ2 hydroxylation is modified relative to the cell or cell-free expression system before introduction of the nucleic acid molecule, and
iv) optionally, selecting a nucleic acid molecule which when expressed increased levels of fatty acid Δ2 hydroxylation.

18. (canceled)

19. A substantially purified and/or recombinant polypeptide which, when expressed in a yeast cell,
i) has an efficiency of conversion of oleic acid to 2-hydroxyoleic acid of at least 1% when compared to a corresponding yeast cell lacking the exogenous nucleic acid,
ii) has an efficiency of conversion of palmitic acid to 2-hydroxypalmitic acid of at least 1% when compared to a corresponding yeast cell lacking the exogenous nucleic acid, and/or
iii) has an efficiency of conversion of palmitoleic acid to 2-hydroxy palmitoleic acid of at least 1% when compared to a corresponding yeast cell lacking the exogenous nucleic acid.

20. The polypeptide of claim 19, wherein the polypeptide comprises amino acids having a sequence as provided in any one of SEQ ID NOs: 1, 2 and 4 to 12, a biologically active fragment thereof, or an amino acid sequence which is at least 30% identical to any one or more of SEQ ID NOs: 1, 2 and 4 to 12.

21. The polypeptide of claim 19, wherein the fatty acid is a C16 or C18 fatty acid.

22. The polypeptide claim 19 which is a fusion protein further comprising at least one other polypeptide sequence.

23. An isolated and/or exogenous polynucleotide comprising:
i) a sequence of nucleotides selected from any one of SEQ ID NOs: 13 to 24,
ii) a sequence of nucleotides encoding a polypeptide of claim 19,
iii) a sequence of nucleotides which are at least 50% identical to one or more of the sequences set forth in SEQ ID NOs:13 to 24, and/or
iv) a sequence which hybridises to any one of i) to iii) under stringent conditions.

24. A chimeric vector comprising the polynucleotide of claim 23, wherein the polynucleotide is operably linked to a promoter.

25. A cell comprising the exogenous polynucleotide of claim 23.

26. A method of producing a polypeptide which, when expressed in a yeast cell,
i) has an efficiency of conversion of oleic acid to 2-hydroxyoleic acid of at least 1% when compared to a corresponding yeast cell lacking the exogenous nucleic acid,
ii) has an efficiency of conversion of palmitic acid to 2-hydroxypalmitic acid of at least 1% when compared to a corresponding yeast cell lacking the exogenous nucleic acid, and/or
iii) has an efficiency of conversion of palmitoleic acid to 2-hydroxy palmitoleic acid of at least 1% when compared to a corresponding yeast cell lacking the exogenous nucleic acid,
the method comprising expressing in a cell or cell free expression system the vector of claim 24.

27. A transgenic non-human organism, or a seed, comprising a cell of claim 1.

28.-29. (canceled)

30. A method of producing seed, the method comprising,
a) growing a plant transgenic non-human organism of claim 27 which is a plant, and
b) harvesting seed from the plant.

31. Oil, or fatty acid, produced by, or obtained from, the cell of claim 1, or a non-human organism comprising said cell.

32. (canceled)

33. An oil comprising fatty acids comprising
i) at least 1.0%, or at least 3.0%, (w/w) 2-hydroxyoleic acid as a percentage of the total fatty acid of the oil,
ii) at least 1.0%, or at least 3.0%, (w/w) 2-hydroxypalmitic acid as a percentage of the total fatty acid of the oil,
iii) at least 0.5% (w/w) 2-hydroxymyristic acid as a percentage of the total fatty acid of the oil, and/or
iv) at least 1.0% (w/w) 2-hydroxy palmitoleic acid as a percentage of the total fatty acid of the oil.

34. A method of producing oil containing Δ2 hydroxylated fatty acids, the method comprising extracting oil from the cell of claim 1, or a non-human organism comprising said cell, wherein at least 1% of the fatty acid content of the oil comprises a 2-hydroxy.

35. (canceled)

36. A fermentation process comprising the steps of:
i) providing a vessel containing a liquid composition comprising a cell of claim 1, or an organism comprising said cell, which is suitable for fermentation, and constituents required for fermentation and fatty acid biosynthesis, and
ii) providing conditions conducive to the fermentation of the liquid composition contained in said vessel.

37. A method of performing a Δ2 hydroxylase reaction, the method comprising contacting a substrate saturated, monounsaturated or polyunsaturated fatty acid with the polypeptide of claim 19.

38. Use of the cell of claim 1, or a non-human organism comprising said cell, for the manufacture of an industrial product.


SEQUENCE LISTING (48 sequences)

SEQ ID NO: 1 - protein, 236 aa, Thymus citriodorus
MVAQGFTVDLNKALVFQVGHLGEDYQEWIHQPIVCKESPRFFENDIIEFLTKNQWWAIPLIWLPVVGWSLSKSISASDAILDVILVTALGVLIWTLMEYSLHRFLFHIETKTYWGNTLHYLLHGCHHKHPMDGLRLVFPPSATAILLYPFWNLIKLFSTPTTGPALFAGILLGYVMYDVTHYYLHHAQPTNEIPKNLKKYHLNHHFRIHDRGFGITSSLWDRVFGTLPQTKAGGKS

SEQ ID NO: 2 - protein, 237 aa, Arabidopsis thaliana
MVAERYTVDLNKPLVFQVGHLGEEYQEWIHQPIVCVEGPRFFESDFWEFLTRTVWWAIPTIWLPVVCYVLSISASKGLTFPQIGLIVAFGVLTWTLLEYTLHRFLFHIQTKSYWANTAHYLLHGCHHKHPQDGLRLVFPPTATAILLVPLWKLLHLLATPATAPAILGGILFGYVMYDITHYYLHHGQPKEPTFKHLKKYHLNHHFRIQDKGYGITSSLWDKVFGTLPGIKAAAKKS

SEQ ID NO: 3 - protein, 237 aa, Arabidopsis thaliana
MVAQGFTVDLKKPLVFQVGHLGEDYEEWVHQPIATKEGPRFFQSDFWEFLTLTVWWAVPVIWLPVVVWCISRSVSMGCSLPEIVPIVVMGIFIWTFFEYVLHRFVFHIKTKSYWGNTAHYLIHGCHHKHPMDHLRLVFPPTATAILCFPFWNIAKAISTPSTAPALFGGGMLGYVMYDVTHYYLHHAQPTRPVTKNLKKYHLNHHFRIQDKGFGITSSLWDIVFGTLPTTKAPRKEQ

SEQ ID NO: 4 - protein, 237 aa, Brassica rapa
MVAERFTVDLDKPLVFQVGHLGEEYQEWIHQPIVCVEGPRFFQSDFWEFLTRTVWWAIPTIWLPVVCYVLSISARKGLTFPQIGLIVAFGVLTWTLLEYTLHRFLFHIETKSYWANTAHYLLHGCHHKHPQDGLRLVFPPTATAILLVPLWKLLHLIATPATAPAMLGGILFGYVMYDVTHYYLHHGQPKEPTFKHLKKYHLNHHFRIQDKGYGITSSLWDRVFGTLPGTKAAQKKL

SEQ ID NO: 5 - protein, 237 aa, Glycine max
MMVVQNFVVDLNKALFFQVGHLGEVYEEWVHQPIVSKEGPRFFENEILEFLTRTVWWAIPVIWLPVVCWFIYKSIRMGLSCPHLALLVVLGIFVWTLLEYSLHRFLFHIKTKTYWGNTLHYLLHGCHHKHPMDGLRLVFPPAATAILLMPFWNLVKLMATTSTAPALFGGGLLGYVMYDCTHYYLHHGQPRTEVPRNLKKYHLNHHFRIQDKGFGITSSLWDKVFGTLPPKMDAKSM

SEQ ID NO: 6 - protein, 239 aa, Glycine max
MVAQKFVVDLNKPLVFQVGHLGEAYDDWVHYPIVSKEGPRFFQSGFLELFTRTAWWVIPTVWVPVASWFISNSVKSGLPSLDVALLVFLGIFVWTLAEYTLHRFLFHVKTKSYWGNTLHYLLHGCHHKHPMDSYRLVFPPVAAAILALPIWNLVKLICTPSVAPAVFGGILLGYVMYDCTHYYLHHGQPKSDVPKSLKKYHLNHHYRLQNYGFGITSPLWDKVFGTVPPPQSKGDAKRR

SEQ ID NO: 7 - protein, 236 aa, Lycopersicon esculentum
MVAQGFTVDLDKPLVHQVGHLGEAYQEWVHQPIVSKEGPRFFASDFWEFLTRTVWWAIPTIWLPVVCYSISVSIRMGRTVPEVALLVVFGIFIWTLMEYTLHRFLFHINTTSYWGNTIHYLLHGCHHKHPMDGLRLVFPPAATAILLVPFWNLIKLLATPTTAPALFGGGLLGYVMYDVTHYYLHHGQPTVEVPKNLKKYHLNHHFRIQNKGFGITSSFWDKVFGTLPHSKSAKNR

SEQ ID NO: 8 - protein, 236 aa, Solanum tuberosum
MVAQGFTVDLDKPLVHQVGHLGEAYQEWVHQPIVSKEGPRFFESDFWEFLTRTVWWAIPTIWLPVVCYSISVSIHMGRTVPEVALMVVFGIFIWTLMEYTLHRFLFHINTRSYWGNTIHYLLHGCHHKHPMDGLRLVFPPAATAILLVPFWNLIKLLATPTTAPALFGGGLLGYVMYDVTHYYLHHGQPTVEVPKNLKKYHLNHHFRIQNKGFGITSSFWDKVFGTLPHSKSAKNR

SEQ ID NO: 9 - protein, 237 aa, Ipomoea nil
MVAQEFTVDLNKPLVFQVGHLGESYQEWVHQPIPSREGPRFFESDFWEFLTRTVWWVIPLVWLPVVCWFVSMSIRMGLTIPHVALHVVFGIFIWTLVEYVLHRFLFHIETKSYWGNTIHYLLHGCHHKHPMDGLRLVFPPAAAAVLSVPFWNLVKLFATPLTAPALFGGGLLGYVMYDVTHYYLHHGQPTSDVPKNLKRYHLNHHFRIQNKGFGITSALWDRVFGTLPPSKTDVKSG

SEQ ID NO: 10 - protein, 236 aa, Nicotiana tabacum
MVAQGFTVDLNKALVFQVGHLGEAYQEWVHQPIVSKEGPRFFESDFWEFLTRTVWWAIPVIWLPVVCYSISVSIWMGHTVPEVALMVVFGIFVWTLMEYTLHRFLFHIKTKSYWANTVHYLLHGCHHKHPMDGLRLVFPPAATAILCVPFWNLIKLMSTPSTAPALFGGGLLGYVMYDVTHYYLHHAQPTSEVPKNLKKYHLNHHFRIQTKGFGITSSFWDKVFGTLPKSKSAKSK

SEQ ID NO: 11 - protein, 237 aa, Oryza sativa
MVVQEFTVDLNKPLVFQVGHLEERYQEWVHQPIVSKEGPRFFGNDVLEFLTRTKWFAVPTIWLPVVCWLLVKSIRMGHTIQEVGLMALFGIFIWTLIEYTLHRFLFHIETKTYWANTAHYLLHGCHHKHPMDSLRLVFPPTATAILCVPFWKLVAFFATPTITPALFGGGLLGYVMYDCTHYYLHHGQPSKEPAKNLKRYHLNHHFRIQNKGFGITSSLWDYVFGTLPPSKTTGKNN

SEQ ID NO: 12 - protein, 237 aa, Triticum aestivum
MVVKEFTVDLNQPLVFQVGHLEEHYQEWVHQPIVSKEGPRFFANDTMEFLTRTKWWAVPVIWLPVVCWLFAKSIRMGHTIQEVILMALFGVFVWTFIEYALHRFLFHIETKSYWSNTAHYLIHGCHHKHPMDSLRLVFPPAGAAIICVPFWNVVAFFASPSTTPALFAGGLLGYVMYDCTHYYLHHGQPSKDPAKHLKRYHLSHHFRIQDKGFGITSSLWDAVFGTLPSSNIVAKLS

SEQ ID NO: 13 - DNA, 710 bp, Thymus citriodorus
atggtggcacagggatttactgttgacttgaataaagcccttgtcttccaggttggccatctcggtgaagattatcaagagtggattcaccagccgattgtctgcaaagaaagccctcgattttttgaaaatgacattattgagtttttgacaaagaatcagtggtgggcgattcctctgatatggctccctgttgtcggttggtctctctcaaagtccatctctgcgagtgatgcaattctcgatgtgatattagtgaccgcacttggtgttttaatctggacattgatggaatattccctacaccgattccttttccacatcgagaccaagacttattggggtaatacacttcattatcttttgcatggctgtcaccataagcacccaatggacggtcttcgcctcgtttttcctccttctgccaccgccattcttttgtatccattctggaacctcattaaattgttcactactcccaccaccggtcctgctctatttgctggtatccttctgggttacgtgatgtacgatgtaactcattactatttgcaccatgcacagccaaccaatgaaataccgaagaatctcaagaaatatcatctaaaccaccatttccgaatccatgacaaaggttttggcattacttcatcgctctgggacagggtgtttggaactctaccccaaacaaaagccggtggaaagagtaa

SEQ ID NO: 14 - DNA, 714 bp, Arabidopsis thaliana
atggttgcagaacgatacacagtcgatttgaataagccacttgtgtttcaggttggtcatcttggcgaagaatatcaggaatggattcatcaacctattgtatgcgttgagggtcctcgttttttcgaaagtgatttttgggagtttttgactcgcacggtttggtgggcaatcccaacaatctggcttccagttgtttgctacgttctttcaatatcggcgagtaagggacttacgtttccccagatcggtttaatagttgcttttggtgtactcacgtggacactgctcgagtacacgcttcatcgcttccttttccacatccaaaccaagagttactgggcaaacactgcacactatcttcttcatggatgccatcacaagcacccacaagacggccttcgtcttgttttccctcctacagcaacagctattctcttggttccactatggaagcttctccatctcctagctactcctgctacagcacctgctattcttggaggcatattgtttggctatgtgatgtatgatattacccattactatctacaccatggtcaacctaaagaaccaacctttaaacatcttaagaaataccacctgaatcatcactttaggattcaggacaaaggatatggaatcacttcctctctatgggacaaagtctttggaacacttcccggtattaaagccgctgcgaagaagagctaa

SEQ ID NO: 15 - DNA, 714 bp, Arabidopsis thaliana
atggttgctcagggattcactgtggatcttaaaaagccccttgtatttcaggttggtcatcttggagaagattatgaggaatgggttcaccaacctatcgcgaccaaggaaggccctcggttttttcagagtgacttttgggagttcttgacacttacagtttggtgggcagttcctgtcatttggttgccagttgtagtctggtgcatatcaaggtcagtaagtatgggatgttcacttccagaaatcgtcccaattgttgtcatgggaatattcatctggacattttttgaatacgttcttcaccggttcgttttccacataaaaacgaagagttactggggaaacactgcacactatcttattcacggatgccatcataagcacccgatggaccaccttcggctcgtctttcctcctactgcaacagcgattttatgctttccgttctggaacattgcgaaggctatctcaactccttcaaccgcacctgcattgtttggtggaggcatgctcggatatgtgatgtacgatgtcactcattattaccttcaccatgcccaacctactagaccagtgaccaaaaatctcaagaagtaccatttgaatcatcacttcaggattcaggacaaaggatttggtataacttcgtcgttatgggacatagtctttgggacacttcccaccacaaaagcccccagaaaagagcaatag

SEQ ID NO: 16 - DNA, 714 bp, Brassica rapa
atggtcgcagaacgcttcacagtcgatttggataagccacttgtctttcaggttggtcatcttggggaagagtatcaggaatggattcatcaacctattgtatgtgtagagggtcctcgttttttccagagtgatttttgggagttcttgactcgcactgtttggtgggcaatcccaacgatctggcttcctgttgtctgctacgttctttcaatctccgcgaggaagggacttacatttccccagatcggtttgatagttgcctttggcgtactcacctggacactgctcgagtacacgcttcatcgtttccttttccacatagaaaccaagagttactgggcaaacactgcacactatcttcttcatggatgccatcacaagcacccacaagacggccttcgtcttgtcttccctcctaccgccacagccattctcttggttccattatggaagcttctccacctcatagctactcctgctacagctcctgctatgcttggaggcattttgtttggctatgtgatgtatgatgttactcattactatctgcaccatggtcaacctaaagaaccaacctttaagcatctcaagaaataccacctgaatcatcacttcagaatccaggacaaaggatatgggatcacttcctctctctgggatagagtctttggaacacttcccggtacaaaggctgctcagaagaagctctaa

SEQ ID NO: 17 - DNA, 714 bp, Glycine max
atgatggttgtgcagaactttgtagttgatttgaataaggccctgtttttccaggttgggcatcttggagaagtttatgaagagtgggttcaccagcccattgtcagcaaggaaggcccccgtttctttgaaaatgagattctggagttcttgacgcgcacagtctggtgggcaataccagtcatttggctgccagttgtatgctggtttatatataagtccatacgaatgggcctcagttgtcctcatttagctctattggtggttcttggcatttttgtttggacactgcttgaatactcattgcatcgttttcttttccacattaaaacaaagacctactgggggaataccttgcattatcttctccatggctgccaccataagcaccccatggatggcttaagactcgttttccctcctgctgcaacagctatattattgatgccgttctggaacttggtgaaactcatggcgaccacttcaactgctcctgctttgtttggaggtggtttattgggttatgtaatgtatgattgcacccattattacttgcaccatggtcagccaaggactgaagtgccccgaaatctcaagaagtaccacttgaatcatcactttcggatccaggataaaggctttgggatcacttcatcactatgggataaagtttttggaacacttcctcctaaaatggatgccaaaagtatgtaa

SEQ ID NO: 18 - DNA, 720 bp, Glycine max
atggttgctcagaaattcgttgtcgacttgaacaagccccttgtattccaggttgggcatcttggggaagcctacgatgattgggttcactatcctatcgtaagcaaagaaggtcctcggtttttccaaagcggtttcctcgagttgttcacacgcactgcatggtgggtgattccaaccgtttgggtgccggttgcgagttggttcatttccaactctgtcaaatcaggcctcccttctcttgatgtagctctgctcgtctttctcggcatctttgtttggactttggctgaatacaccttgcaccgtttcctcttccatgttaaaaccaagagctactggggaaataccttgcattatcttctccatggctgccaccacaagcaccccatggatagctatagacttgttttcccccctgtagcagcagctatattagccctcccgatctggaacttggtgaagctgatatgcaccccttcagttgctcctgcggtgtttggaggtattttgttgggctatgtgatgtatgattgcacccattactacttacaccatggtcaaccaaagtctgatgtgcccaaaagtctcaagaaatatcacttgaatcatcattatcggctccagaactatggctttgggatcacatcaccactgtgggataaagtttttggaacggttcctcctcctcaatcaaagggggatgccaaacgtagataa

SEQ ID NO: 19 - DNA, 711 bp, Lycopersicon esculentum
atggttgcacaaggcttcactgtggatttagataagccacttgtacaccaggttggccaccttggagaagcttatcaagagtgggttcaccagcccattgtcagcaaggaaggtcctcgattttttgctagtgacttttgggagtttctgactcgcacagtttggtgggcaattcctaccatatggcttccagttgtgtgctatagcatctctgtttctatccgtatgggtcgtactgttccagaggtggcactgttggtggtttttggcattttcatctggacactgatggaatacactcttcatcgttttcttttccatatcaatacaacgagctattggggaaacacaattcattatcttcttcatggctgtcatcataagcatcccatggatggtctacggctggtttttcctcctgctgcaacggctattctcctcgtaccattctggaacttgatcaaattgctggcaactcccactactgctcctgctttgtttggaggcggtttattgggctatgttatgtatgatgtaacccactactacttgcatcacgggcaaccaacagttgaagtgcctaaaaatctcaagaaatatcacttgaatcatcattttcgtatccagaacaaaggttttggtatcacctcttccttctgggacaaagtgtttggaactctaccacattcaaaatcagccaagaacagatga

SEQ ID NO: 20 - DNA, 711 bp, Solanum tuberosum
atggttgcacaaggcttcactgtggatttagataagccacttgtacaccaggttggccaccttggagaagcttatcaagagtgggttcaccagcccattgtcagcaaggaaggtcctcgattttttgaaagtgatttttgggagtttctgactcgcacagtttggtgggcaattcctaccatatggcttccagttgtgtgctatagcatctctgtatctatccatatgggtcgtactgttccagaggtggcactgatggtggtttttggcattttcatctggacactgatggaatacactcttcatcgttttcttttccacatcaatacaaggagctattggggaaacactattcattatcttcttcatggctgtcatcataagcatcccatggatggtctacggctggtttttcctcctgctgcaacagctattctccttgtaccattctggaacttgatcaaactcctggcaactcccactactgctcctgctttgtttggaggcggtttattgggctatgttatgtatgatgtaacccactactacttgcatcacgggcaaccaacagttgaagtgcctaaaaatcttaagaaatatcacttgaatcatcattttcgtattcagaacaaaggttttggtatcacctcctccttctgggacaaagtctttggaacgctaccacattcaaaatcagccaagaacagatga

SEQ ID NO: 21 - DNA, 714 bp, Ipomoea nil
atggttgcacaggaattcacggtggatctgaataaacccctcgtcttccaggttggccatcttggagaatcttatcaggagtgggttcatcagcctattcctagcagagaaggtcctcggttttttgaaagtgacttttgggagtttctcacccgcactgtgtggtgggtaattcctctcgtttggctcccagttgtatgctggttcgtttcaatgtctattcggatgggtctcacgattcctcatgttgcactacatgtagtatttggcattttcatttggacactagtggaatacgttcttcaccgcttcctttttcatattgagacaaagagctattgggggaataccattcattatcttcttcatggttgccaccacaagcaccccatggatggactgcgccttgttttccctcctgctgcagcagctgttctcagtgtgccattctggaacctagtaaagctctttgccactccattgactgctcctgctttgttcggaggaggcttactgggttatgtgatgtatgatgtaacccattactacttgcaccatggacaacccacgagtgatgtgccaaaaaatctcaagagatatcacctgaaccaccattttcgtatccagaacaaaggtttcggcattacttccgcattgtgggatagagtgtttggaacgttaccaccatcaaaaacagatgtgaagagtggataa

SEQ ID NO: 22 - DNA, 711 bp, Nicotiana tabacum
atggttgcacagggcttcactgtggatttaaataaagctctcgtcttccaggttggccaccttggagaagcttatcaagaatgggttcatcagcctattgttagcaaggaaggacctcgattttttgaaagtgatttttgggagtttctgactcgcacagtttggtgggcaattccagtcatttggctcccagttgtatgctatagtatctcagtatcaatttggatgggccatactgttcccgaggtggcattgatggtagtttttggaatttttgtgtggacattgatggaatatactcttcatcgattcctgttccatataaagacgaagagctattgggcaaacacggttcattatcttcttcatggttgtcatcacaagcatcctatggatggtctacgccttgtttttcctcctgctgccacagctattctttgtgtaccgttctggaatttaatcaaactcatgtcaactccctctactgctcctgccttatttggaggaggcttattgggctatgtcatgtatgatgttacacactactacttgcatcatgcacaaccaacaagtgaagtgccaaaaaatctcaagaaatatcatttgaatcatcattttcgcatccagaccaaaggttttggtatcacttcctctttctgggacaaggtgtttgggacgctaccaaaatcgaaatcagccaagagcaaatga

SEQ ID NO: 23 - DNA, 714 bp, Oryza sativa
atggttgtccaggagttcaccgttgatctgaacaagcctctcgttttccaggttggccatcttgaggaacgttatcaggaatgggttcatcaacctatcgtgagcaaggaaggtccacgctttttcggaaatgatgtcctggagttcttgacgcgcacaaagtggtttgctgtgccaaccatatggctgcctgtcgtctgctggttgcttgtgaaatctattcggatgggtcatactattcaggaagtaggtctaatggccctatttggaatattcatttggacattgatcgagtatactttgcaccgcttcctattccacattgagaccaaaacctattgggcaaacacagcacattaccttcttcatggatgccaccataagcatcctatggactcactcaggcttgtattcccccctactgcaacagcaattttgtgtgtgccgttctggaagctggtagcgttcttcgcaactccaactatcactcctgcgctatttggtggtggcctgttgggatatgtgatgtatgactgcactcactattacttgcaccatggacaaccatccaaagagccagcgaaaaatctcaagcggtatcacctcaaccaccacttcagaatccaaaacaaaggctttggcatcacctcatcactctgggattacgtttttgggacattgcctccgtcgaagaccaccggaaagaacaactga

SEQ ID NO: 24 - DNA, 714 bp, Triticum aestivum
atggttgtcaaggagttcaccgttgatctcaaccagcctcttgttttccaggttggccatctcgaggaacattaccaggaatgggttcaccagcctatcgtcagcaaagaaggtccgcgctttttcgcaaatgataccatggagttcttgacacgcacaaagtggtgggctgtgccagtcatatggctacctgtcgtctgctggctgtttgccaagtcgattcggatgggtcacaccattcaggaagtaatcctgatggccctatttggagtgtttgtttggacgttcattgagtacgctttgcaccgcttccttttccacatcgagacgaaaagctattggtcgaacaccgcacactaccttatccatggatgccaccataagcatcctatggactcactcaggctcgtattccctcctgctggggcagcgatcatatgtgtgccgttctggaatgttgtggcattcttcgccagtccatctaccactcctgcgctgttcgccggtggcctgctggggtacgtgatgtatgactgcacgcactactacctgcaccatggacagccatccaaagatccagcgaagcatctcaagcggtaccacctcagccaccatttcagaatccaggacaagggcttcggcatcacctcgtcgctctgggacgccgtttttgggacgctgccttcatccaatatcgttgcgaagctcagctga

SEQ ID NO: 25 - DNA, 19 bp, Artificial Sequence
  Oligonucleotide primer
ttccgtctcgagtcgctga

SEQ ID NO: 26 - DNA, 29 bp, Artificial Sequence
  Oligonucleotide primer
ctttctagactattgctcttttctggggg

SEQ ID NO: 27 - DNA, 29 bp, Artificial Sequence
  Oligonucleotide primer
tttggatcctctaagaaacaatcagtctc

SEQ ID NO: 28 - DNA, 28 bp, Artificial Sequence
  Oligonucleotide primer
gcatctagaaatccgatttagctcttct

SEQ ID NO: 29 - DNA, 41 bp, Artificial Sequence
  Oligonucleotide primer
gtngayctnrayaarccnctngtnttycargtnggncayct

SEQ ID NO: 30 - DNA, 41 bp, Artificial Sequence
  Oligonucleotide primer
tncaycarccnathgtntcnaargarggnccncgnttytty

SEQ ID NO: 31 - DNA, 30 bp, Artificial Sequence
  Oligonucleotide primer
gtgggcnathccnacnathtggctnccngt

SEQ ID NO: 32 - DNA, 47 bp, Artificial Sequence
  Oligonucleotide primer
tggacnctnatggartayacnctncaycgnttyctnttycayathaa

SEQ ID NO: 33 - DNA, 54 bp, Artificial Sequence
  Oligonucleotide primer
ccaatanganttngtntttatatgaaanagaaancgatgnagngtatattccat

SEQ ID NO: 34 - DNA, 35 bp, Artificial Sequence
  Oligonucleotide primer
caytayctnctncayggntgycaycayaarcaycc

SEQ ID NO: 35 - DNA, 35 bp, Artificial Sequence
  Oligonucleotide primer
ggrtgyttrtgrtgrcanccrtgnagnagrtartg

SEQ ID NO: 36 - DNA, 29 bp, Artificial Sequence
  Oligonucleotide primer
agdatngcngcngcngcngcnggnggraa

SEQ ID NO: 37 - DNA, 44 bp, Artificial Sequence
  Oligonucleotide primer
ggytgnccrtgrtgnagrtartartgngtnacrtcrtacatnac

SEQ ID NO: 38 - DNA, 36 bp, Artificial Sequence
  Oligonucleotide primer
ytgdatncgraartgrtgrttnagrtgrtayttytt

SEQ ID NO: 39 - DNA, 33 bp, Artificial Sequence
  Oligonucleotide primer
rtcccaraangangangtdatnccraanccytt

SEQ ID NO: 40 - protein, 14 aa, Arabidopsis thaliana
  MISC_FEATURE (4)..(4): X = D or N
VDLXKPLVFQVGHL

SEQ ID NO: 41 - protein, 15 aa, Arabidopsis thaliana
WVHQPIVSKEGPRFF

SEQ ID NO: 42 - protein, 11 aa, Arabidopsis thaliana
WWAIPTIWLPV

SEQ ID NO: 43 - protein, 21 aa, Arabidopsis thaliana
WTLLEYTLHRFLFHIKTKSYW

SEQ ID NO: 44 - protein, 12 aa, Arabidopsis thaliana
HYLLHGCHHKHP

SEQ ID NO: 45 - protein, 13 aa, Arabidopsis thaliana
  MISC_FEATURE (10)..(10): X = D or N
RLVFPPAAAXAIL

SEQ ID NO: 46 - protein, 17 aa, Arabidopsis thaliana
GYVMYDCTHYYLHHGQP

SEQ ID NO: 47 - protein, 12 aa, Arabidopsis thaliana
KKYHLNHHFRIQ

SEQ ID NO: 48 - protein, 11 aa, Arabidopsis thaliana
KGFGITSSLWD